(19) United States
(12) Patent Application Publication
(10) Pub. No.: US 2020/0181095 A1
(43) Pub. Date: Jun. 11, 2020

(54) SELECTIVE MATRIX METALLOPROTEINASE-13 INHIBITORS

(71) Applicants: FLORIDA ATLANTIC UNIVERSITY BOARD OF TRUSTEES, Boca Raton, FL (US); THE SCRIPPS RESEARCH INSTITUTE, La Jolla, CA (US)

(72) Inventors: Gregg B. FIELDS, Palm Beach Gardens, FL (US); William R. ROUSH, Jupiter, FL (US); Jun Yong CHOI, La Jolla, CA (US); Rita FUERST, La Jolla, CA (US)

(73) Assignees: Florida Atlantic University Board of Trustees, Boca Raton, FL (US); The Scripps Research Institute, La Jolla, CA (US)

(21) Appl. No.: 16/619,737

(22) PCT Filed: Jun. 6, 2018
(86) PCT No.: PCT/US2018/036266

§ 371 Date: Dec. 5, 2019

Related U.S. Application Data
(60) Provisional application No. 62/515,793, filed on Jun. 6, 2017.

(51) Int. Cl. C07D 239/70; C07D 405/12

(52) U.S. Cl. CPC C07D 239/70; C07D 405/12

(57) ABSTRACT

We describe the use of comparative structural analysis and structure-guided molecular design to develop potent and selective inhibitors (10d and (S)-17b) of matrix metalloproteinase 13 (MMP-13). We applied a three-step process, starting with a comparative analysis of the X-ray crystallographic structure of compound 5 in complex with MMP-13 with published structures of known MMP-13 inhibitor complexes followed by molecular design and synthesis of potent, but non-selective zinc-chelating MMP inhibitors (e.g., 10a and 10b). After demonstrating that the pharmacophores of the chelating inhibitors (S)-10a, (R)-10a, and 10b were binding within the MMP-13 active site, the Zn2+ chelating unit was replaced with non-chelating polar residues that bridged over the Zn2+ binding site and reach into a solvent accessible area. After two rounds of structural optimization, these design approaches led to small molecule MMP-13 inhibitors 10d and (S)-17b which bind within the substrate-binding site of MMP-13 and surround the catalytically active Zn2+ ion without chelating to the metal. These compounds exhibit at least 500-fold selectivity versus other MMPs.

### CLAIM OF PRIORITY

This application claims the benefit of priority to U.S. Provisional Application Ser. No. 62/515,793, filed on Jun. 6, 2017, which is incorporated herein by reference in its entirety.

### STATEMENT OF GOVERNMENT SUPPORT

This invention was made with government support under AR063795 awarded by the National Institutes of Health. The government has certain rights in the invention.

### BACKGROUND

Matrix Metalloproteinase-13 (MMP-13) is known to be mainly responsible for the cleavage of type II collagen in osteoarthritis (OA).^{1,2} The expression of MMP-13 is highly upregulated (>40-fold) in the cartilage of OA patients, but is hardly detectable in healthy individuals.' Recent reports demonstrate that MMP-13 activity is involved in inflammatory bowel diseases as well as melanoma cell invasion and breast cancer metastasis, which make MMP-13 an even more interesting therapeutic target.⁴⁻⁶

The 24-membered MMP family is highly conserved, with sequence similarity between 56 and 64% in their active domains.⁷ The common structural element in the MMP active site is a Zn²⁺ ion coordinated by a tris(hisfidine) motif.⁸ The first WNW inhibitors, discovered in the 1990s, were not selective for any particular MMP because of their zinc-chelating functional units.^{9,10}

Several of these compounds entered clinical trials but all were withdrawn due to the occurrence of musculoskeletal toxicities evoked by unselective binding within the MMP family.¹¹⁻¹³ More recently, a selective Zn-binding inhibitor containing a 1,2,4-triazole ring as the metal coordinating group showed promising results in the inhibition of collagen release from cartilage in vitro.¹⁴ A more detailed analysis of the MMP active site led to the discovery of six subsites (S1-S3 and S1′-S3′) surrounding the catalytic Zn²⁺ ion.¹⁵ Of these, the S1′ subsite is surrounded by a specificity loop (Ω-loop), which encloses the so-called S1′* specificity pocket and varies in the length and amino acid sequence for different MMP isoforms.¹⁵ Targeting Lys140, which is unique at the bottom of the S1′* subsite of MMP-13 vs. other MMP isozymes, has provided the basis for the development of highly selective MMP-13 inhibitors. Consequently, various agents possessing a benzoic acid unit, which can form a salt bridge interaction with Lys140, have emerged as highly specific MMP-13 inhibitors (1-4, FIG. 1).¹⁶⁻¹⁸ However no MMP-13 inhibitor has yet received FDA approval. Some of the most promising recent selective MMP-13 inhibitors had poor solubility, permeability, biodistribution, metabolic stability, and/or bioavailability and thus the search for new MMP-13 inhibitors continues.¹⁹

### SUMMARY

In various embodiments, the invention provides a selective matrix metalloproteinase-13 inhibitor of formula

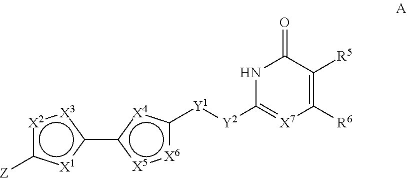

wherein

group Z is of formula S(O)₂NR^{1A}R^{1B} or of formula C(═O)NHCH(R^{2A})C(═O)NHR^{2B};

R^{1A} is H, and R^{1B} is (C₁-C₄)alkyl, HO₂C—(C₁-C₄)alkyl, HO—(C₁-C₄)alkyl, or H₂N—(C₁-C₄)alkyl; or is (C₃-C₇)cycloalkyl, HO₂C—(C₃-C₇)cycloalkyl, HO—(C₃-C₇)cycloalkyl, or H₂N—(C₃-C₇)cycloalkyl; or is 5- to 7-membered heterocyclyl optionally substituted with HO₂C—, HO—, or H₂N—; or is (C₆-C₁₀)aryl optionally substituted with HO₂C—, HO—, or H₂N—; or is 5- to 7-membered heteroaryl optionally substituted with HO₂C—, HO—, or H₂N—;

R^{2A} is (C₁-C₄)alkyl, (C₃-C₂)cycloalkyl, 5- to 7-membered heterocyclyl, (C₆-C₁₀)aryl, or 5- to 7-membered heteroaryl, and R^{2B} is H, (C₁-C₄)alkyl, (C₃-C₇)cycloalkyl, 5- to 7-membered heterocyclyl, (C₆-C₁₀)aryl, or 5- to 7-membered heteroaryl;

X¹ is CH, O, S, C(R³)═C(R³), NIR³, or N=C(R³);

X² and X³ are each independently O, S, N or CR³;

such that the ring comprising X¹, X², and X³ is aryl or heteroaryl;

R³ is independently at each occurrence H, (C₁-C₄)alkyl, or halo;

X⁴ is CH, O, S, C(R⁴)═C(R⁴), NIR⁴, or N=CR⁴;

X⁵ and X⁶ are each independently O, S, N or CR⁴;

such that the ring comprising X⁴, X⁵, and X⁶ is aryl or heteroaryl;

R⁴ is independently at each occurrence H, (C₁-C₄)alkyl, or halo;

Y¹ is CHR, O, NR, or a bond;

Y² is S, CHR, NR, or O, or a bond;

X⁷ is N or CR;

R is H or (C1-C4)alkyl;

R⁵ and R⁶ are each independently H, (C₁-C₄)alkyl, or halo; or R⁵ and R⁶ together with the ring carbon atoms to which they are bonded together form a 5- to 7-membered cycloalkyl ring or a 5- to 7-member heterocyclyl ring;

or a pharmaceutically acceptable salt thereof.

For instance, for the compound of formula A, X¹ and X⁴ can both be CH═CH.

For instance, for the compound of formula A, X² and X³ can both be CR³.

For instance, for the compound of formula A, X⁵ and X⁶ can both be CR⁴.

For instance, for the compound of formula A, X¹ can be O and X⁴ can be CH═CH.

For instance, for the compound of formula A, R³ and R⁴ are all H. Alternatively, at least one R⁴ can be F.

For instance, for the compound of formula A, X¹ can be CH═CH, and X² and X³ can both be CH; more specifically, the compound can be any of the compounds of formula 10, shown in Table 1, below,

For instance, for the compound of formula A, X¹ can be O, X² and X³ can both be CH; and R⁴ can be H or F; more specifically the compound can be any of the compounds of formula 17, shown in Table 2, below.

In various embodiments, the invention provides a pharmaceutical composition comprising a compound of formula A, and a pharmaceutically acceptable excipient.

In various embodiments, the invention provides a method of selective inhibition of MMP-13 comprising contacting MMP-13 with an effective amount or concentration of a compound of formula A, or a pharmaceutically acceptable salt thereof.

In various embodiments, the invention provides a method of treatment of a disease in which selective inhibition of MMP-13 is indicated, such as for treatment of OA, inflammatory bowel disease, melanoma, or breast cancer, comprising administering to a patient afflicted therewith an effective dose of a compound of formula A, or a pharmaceutically acceptable salt thereof.

### BRIEF DESCRIPTION OF THE DRAWINGS

FIG. 1. Highly selective MMP-13 inhibitors 1-4.

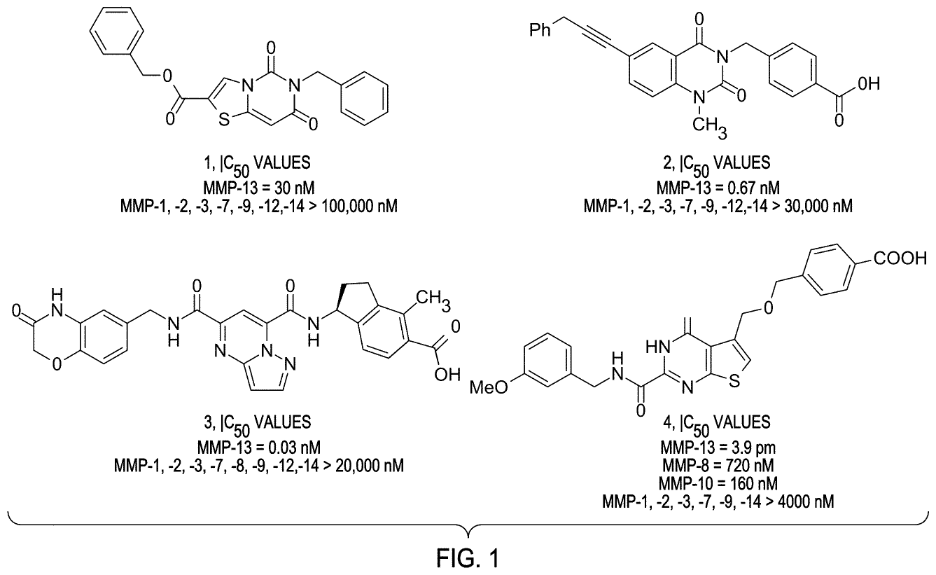

FIGS. 2A-C, Comparative structural analysis and design of Zn-chelating inhibitors. (A) X-ray crystallographic structure of MMP13⋅5 complex (PDB ID: 4L19), Hydrogen bond interactions of 5 with the amide backbone units of Thr245 and Thr247 are represented in black dashed lines. Leu239, Phe252, and Pro255 form hydrophobic contacts with 5, (B) The 4-methylphenyl ring of 5 is oriented toward the Zn binding site and the MMP-13 S1 subsite. (C) The cyclopentyl ring of 5 occupies the S1′ subsite of MMP-13.

FIG. 3. Inhibition of collagen cleavage activity of MMP-13. Inhibition was determined as a percent of intact type II collagen remaining after 24 hours of incubation at 37° C. Collagen oligomers are observed at >250 kD.

FIG. 4, Results of compound selectivity against MMP isozyme panel. Compounds 5, (S)-10a, (R)-10a and 10b were tested at a single concentration of 20 μM. The inhibition of each isozyme was determined as a percent conversion of the substrate to product after 30 min of incubation.

FIG. 5. Promiscuity assay of (S)-17a against MMP isozymes. The inhibition of each isozyme was determined as a percent conversion of the substrate to product after 30 min of incubation. The compounds (S)-17a was tested against the MMP isozyme panel at a single concentration of 200 nM.

### DETAILED DESCRIPTION

Structure guided drug design has been increasingly utilized in modern drug discovery and provides many opportunities for the rational development of drug candidates. Indeed, the rapidly expanding number of protein X-ray structures constitutes a significant resource of structural information useful for structure-guided drug design and has greatly facilitated the drug discovery processes. ²⁰⁻²²

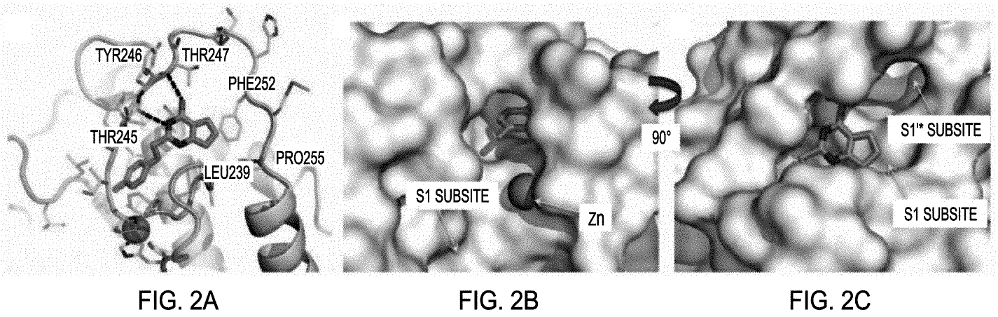

MMP-13 in complex with 5 (FIGS. 2A, 2B, and 2C)²³ and synthesis of 10a-f. We designed MMP-13 inhibitors based on our previously published MMP-13⋅5²³ complex with multiple MMP-13⋅inhibitor crystallographic structures currently available in the Protein Data Bank (PDB)²⁴.

Compounds (S)-10a, (R)-10a and achiral 10b were synthesized by using the synthetic route outlined in Scheme 1. Treatment of 4-(bromomethyl)biphenyl (7) with the thiopyrimidinone fragment 8²⁵ in DMF in the presence of triethyl amine provided 9 in high yield. Chlorosulfonation of 9 followed by treatment with either L- or D-valine or glycine as the nucleophile gave the chelating MMP-13 inhibitors (S)-10a, (R)-10a and 10b, respectively.

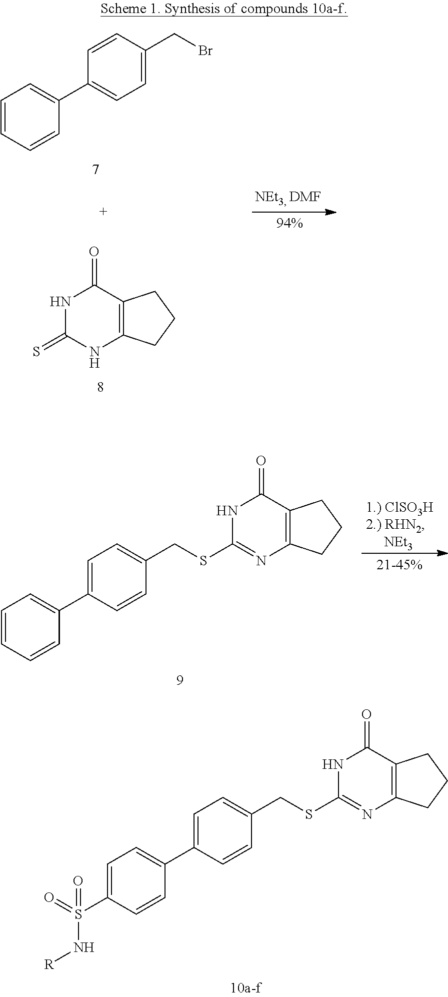

All three compounds displayed significant inhibition potency toward MMP-13 (IC₅₀ values of 2.2, 2.4, and 1.6 nM, respectively) with inhibition constants (Kᵢ) of 2.3, 1.6, and 1.8 nM, respectively (Table 1). This constituted an almost 1000-fold improvement of inhibition potency compared to the starting inhibitor (5).

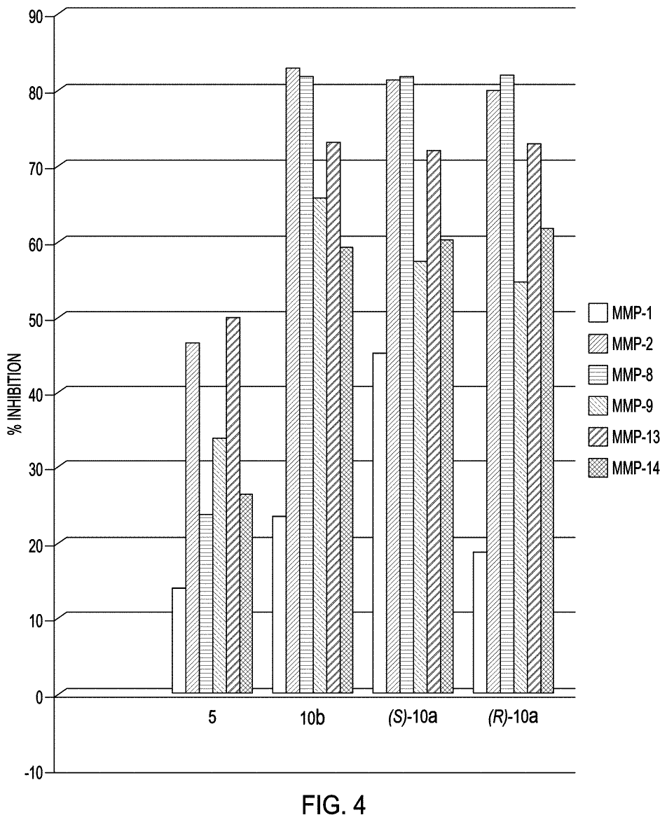

The biological data for compounds 10a-f are shown in Table 1. The selectivity of compounds 5 as well as (S)-10a, (R)-10a, and 10b for MMP-1, -2, -8,-9, and -14 were only tested at a single concentration and the % inhibition results are shown in FIG. 4.

TABLE 1
IC₅₀, Kᵢ values and selectivity data for 5 and 10a-f.

| | | | Kᵢ | | | | | | |
|---|---|---|---|---|---|---|---|---|---|
| | | Inhibitor | MMP- | IC₅₀ (nM)^{a} | | | | | |
| | R—NH₂ | Type | 13 | MMP-13 | MMP-1 | MMP-2 | MMP-8 | MMP-9 | MMP-14 |
| 5 | — | | 800 | 2400 | —^{b} | —^{b} | —^{b} | —^{b} | —^{b} |
| (S)-10a | 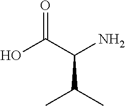 | Zn²⁺ chelator | 2.3 | 2.2 | —^{c} | —^{c} | —^{c} | —^{c} | —^{c} |
| (R)-10a | 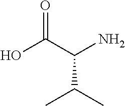 | Zn²⁺ chelator | 1.6 | 2.4 | —^{c} | —^{c} | —^{c} | —^{c} | —^{c} |
| 10b | 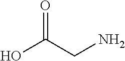 | Zn²⁺ chelator | 1.8 | 1.6 | —^{c} | —^{c} | —^{c} | —^{c} | —^{c} |
| 10c | 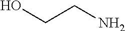 | non- chelator | 12 | 9.2 | 4000 | >5000 | >5000 | >10000 | >10000 |
| 10d | 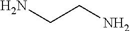 | non- chelator | 13 | 3.4 | >5000 | 730 | 600 | >10000 | >10000 |
| 10e | 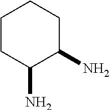 | non- chelator | — | 18 | >10000 | 2700 | 3500 | >10000 | >10000 |
| 10f | 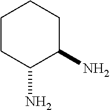 | non- chelator | — | 18 | >10000 | >10000 | >10000 | >10000 | >10000 |

^{a}The IC₅₀ values were determined by using fluorescence resonance energy transfer triple-helical peptides (fTHPs) as substrates in the enzyme assay.^{23,26,27}
^{b}Compound 5 was tested against MMP-1, -2, -8, -9, and -14 at a single concentration and these data are reported in reference 25 (Roth et. al.).
^{c}Since (S)-10a, (R)-10a, and 10b are expected to be Zn-chelating agents, these were only tested at a single concentration (Figure S1).

To assess the selectivity among the MMP family we tested all compounds for their inhibition of MMP-1, -2, -8, -9, and -14, which are the close relatives of MMP-13 with sequence homologies higher than 60% and which are also capable of cleaving different types of collagen.^{7,28-30} Triple-helical peptides (THPs) containing a fluorophore and a quencher within the same peptide chain were used as enzyme substrates, whereby fluorescence resonance energy transfer (FRET) measurements assessed enzymatic conversion.27 Due to the conformational features of THPs, the interaction with MMP subsites is more precise than in the case of single-stranded substrates.^{26,31}

Compounds 10c and 10d proved to be potent MMP-13 inhibitors, with 9.2 and 3.4 nM IC₅₀'s, respectively (Table 1). The selectivity of both compounds toward other MMP isozymes was significantly improved compared to the Zn-chelating inhibitors 10a-b (Table 1). The inhibition potency of 10c toward MMP-1, -2, -8, -9 and -14 was more than 400-fold weaker compared to MMP-13. Compound 10d inhibits MMP-2 and MMP-8 with IC₅₀ values of 730 nM and 600 nM, respectively, but does not inhibit MMP-1, -9, and -14 at the highest concentration tested (10 μM).

Compounds 10e and 10f are marginally weaker MMP-13 inhibitors (IC₅₀=18 nM for both) compared to 10d but also exhibit high selectivity against other MMP isozymes (MMP-1, -2, -8, -9, and 14), as shown by the data in Table 1. Inhibitor 10f has IC₅₀>10,000 nM against all five of these other MMP's, while 10e exhibits weak inhibition of MMP-2 and MMP-8 with IC₅₀ values of 2.7 μM and 3.5 μM, respectively.

Compounds 17a-c were also synthesized (Scheme 2). We intended to keep the core of 5 as part of a second-generation set of inhibitors and to replace the phenyl-sulfonamide moiety in 10a.

The Suzuki coupling reaction of bromofuran 12 and arylboronic acids 13 and 14 yielded the expected biaryl fragments, which were subsequently converted into the benzylic bromide intermediates 15. After alkylation of 15 with the thiopyrimidinone fragment 4, syntheses of 17a-c were completed following ester hydrolysis and amide formation.

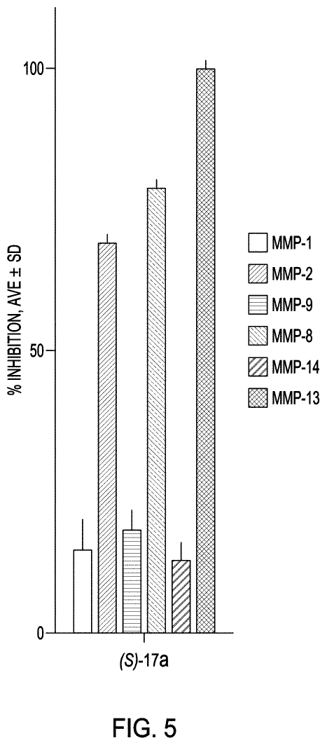

The biological data of compounds 17a-e arc shown in Table 2. inhibitor (S)-17a was tested against MMP-1, m2, -8, -9, and -14 at a single concentration and data are reported in FIG. 5.

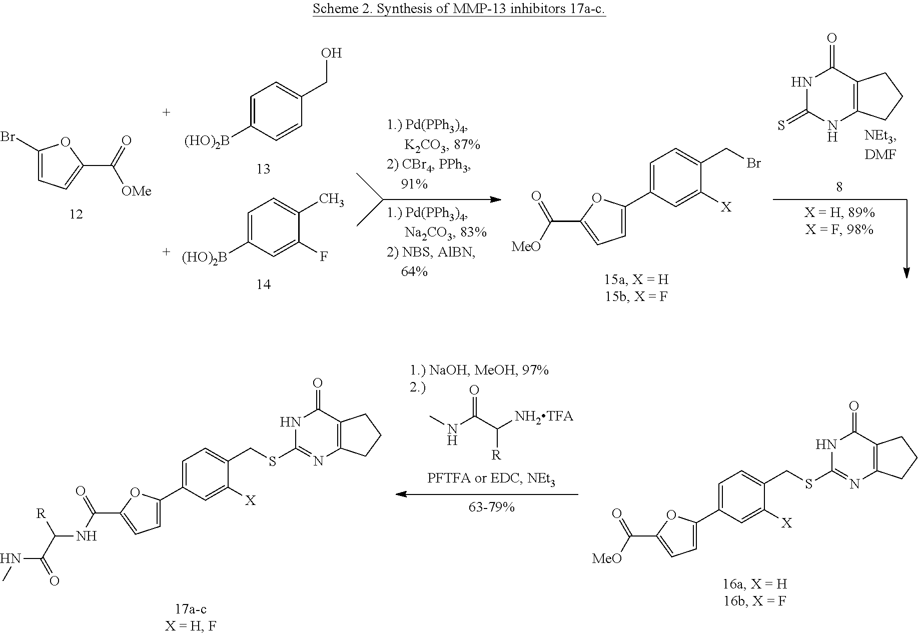

TABLE 2
IC₅₀ values and selectivity data for 17a-17c.

| | | | IC₅₀ (nM)^{a} | | | | | |
|---|---|---|---|---|---|---|---|---|
| | | | MMP- | MMP- | MMP- | MMP- | MMP- | MMP- |
| | X | R | 13 | 1 | 2 | 8 | 9 | 14 |
| (S)- 17a | H | 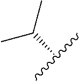 | 5.9 | —^{b} | —^{b} | —^{b} | —^{b} | —^{b} |
| (R)- 17a | H | 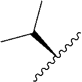 | 72 | —^{c} | —^{c} | —^{c} | —^{c} | —^{c} |
| (S)- 17b | F | 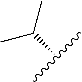 | 2.7 | >5000 | >5000 | >5000 | >5000 | >5000 |
| (R)- 17b | F | 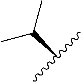 | 257 | >5000 | 3100 | >5000 | >5000 | >5000 |
| (S)- 17c | F | 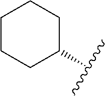 | 4.4 | >5000 | >5000 | >5000 | >5000 | >5000 |
| (R)- 17c | F | 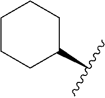 | 159 | >5000 | >5000 | >5000 | >5000 | >5000 |

^{a}The IC₅₀ values were determined using fluorescence resonance energy transfer triple-helical peptides (fTHPs) as substrates in the enzyme assay.^{23,26,27}
^{b}Inhibitor (S)-17a was tested against MMP-1, -2, -8, -9, and -14 at a single concentration and data are reported in Figure S2.
^{c}Not tested due to decrease in potency compared to (S)-17a.

The inhibition potency of (5′)-17a (IC₅₀=5.9 nM) vs MMP-13 was nearly 1,000-fold improved compared to 5. However, (5)-17a proved to be a moderately active inhibitor of MMP-2 and MMP-8 when tested at 200 nM in a single dose assay.

Replacement of the L-valine unit (in (S)-17a) with the unnatural amino acid D-valine (to give (R)-17a) resulted in a ca. 12-fold loss of inhibition activity vs. MMP-13 (Table 2). Compound (S)-17b, with an ortho-fluorophenyl ring, exhibited improved selectivity for MMP-13 compared to (S)-17a, while retaining its inhibition potency (Table 2). The introduction of the unnatural amino acid D-valine ((R)-17b) resulted in a drop of activity by almost 100-fold compared to (S)-17b. Furthermore, (S)-17e was a low nanomolar MMP-13 inhibitor (IC₅₀=4.4 nM) and had an excellent selectivity profile, with >1,000-fold selectivity when tested against the MMP isozymes. Again, enantiomeric (R)-17c lost activity toward MMP-13 (IC₅₀=159 nM) but still exhibited a very clean selectivity profile within the collagenases MMP-1-2, -8, -9, and -14.

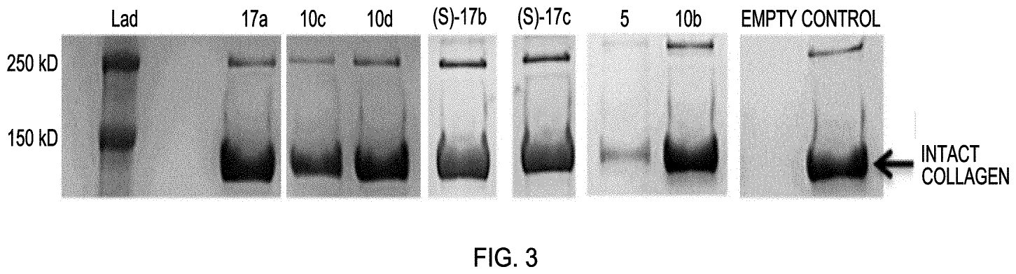

inhibition of type II collagen cleavage. The potential of inhibitors 10c-f and 17a-e for modifying the degradation of articular cartilage by MMP-13 was evaluated in an in vitro type 11 collagen cleavage assay,³² These compounds exhibited >90% inhibition of collagenolysis at 20 mM, while 5 is nearly inactive at this concentration (FIG. 3). Further analysis revealed that the highly selective MMP-13 inhibitors 10d, (S)-17b, and (S)-17c possess low nM inhibition potency (IC₅₀=8.3, 8.1 and 7.9 nM, respectively) against the collagen cleavage activity of MMP-13.

Specificity Profiling of 10d and (S)-17b. The protease selectivity of highly potent MMP-13 inhibitors (10d and (S)-17b) was evaluated by using a profiling assay against 25 proteases (Table 3). As expected, 10d and (S)-17b exhibited high inhibition potency vs. MMP-13 (97% at 1 μM) in this profiling assay, but were substantially if not entirely inactive vs. most other proteases tested. Interestingly, 10d is a modestly active inhibitor of MMP-12 (40% inhibition at 1 μM), while (S)-17b is moderately active against MMP-3 and MMP-12 with 63% and 81% inhibition, respectively, at 1 μM. Subsequently, the inhibition IC₅₀ values for 1.0d and (S)-17b vs. MMP-3 and MMP-12 were determined in a 10-point dilution assays using FRET single-stranded peptide substrates.³³ These determinations established that the IC₅₀ values for 10d and (S)-17b as inhibitors of MMP-12 are 470 nM and 1,800 nM, respectively (e.g., 140- and 700-fold less active than their activity as MMP-13 inhibitors).

TABLE 3
Protease selectivity profiling for 10d and (S)-17b.

% Inhibition^{a}

| Enzyme | 10d | (S)-17b |
|---|---|---|
| ACE | 7 | 0 |
| ACE2 | 1 | 0 |
| ADAM10 | 0 | 0 |
| BACE-1 | 1 | 0 |
| Caspase-1 | 1 | 0 |
| Caspase-2 | 0 | 0 |
| Caspase-3 | 0 | 0 |
| Caspase-5 | 0 | 0 |
| Caspase-6 | 0 | 0 |
| Caspase-7 | 1 | 8 |
| Cathepsin-D | 3 | 0 |
| Cathepsin-K | 0 | 2 |
| Cathepsin-L | 9 | 7 |
| Cathepsin-S | 12 | 2 |
| Factor-XA | 3 | 0 |
| Furin | 0 | 0 |
| IDE | 0 | 0 |
| MMP-3 | 13 | 63 (IC₅₀ = 4.4 μM)^{b} |
| MMP-7 | 7 | 4 |
| MMP-12 | 40 (IC₅₀ = 467 nM)^{b} | 81 (IC₅₀ = 1.8 μM)^{b} |
| MMP-13 | 97 | 97 |
| Neprilysin | 3 | −6 |
| TACE | 4 | −3 |
| Thrombin | −3 | −8 |
| uPA | 3 | 1 |

^{a}% Inhibition was determined by using single stranded peptide substrates at an inhibitor concentration of 1 mM. Assays were performed in duplicates, % inhibition was determined, and average values are present.
^{b}Additional specificity assays against MMP-3 and MMP-12 were performed using fTHP substrates to determine the IC₅₀ values.

### Definitions

Cycloalkyl groups are groups containing one or more carbocyclic ring including, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups also include rings that are substituted with straight or branched chain alkyl groups.

Aryl groups are cyclic aromatic hydrocarbons that do not contain heteroatoms in the ring. An aromatic compound, as is well-known in the art, is a multiply-unsaturated cyclic system that contains 4n±2π electrons where n is an integer.

Heterocyclyl groups or the term “heterocyclyl” includes aromatic and non-aromatic ring compounds containing 3 or more ring members, of which one or more ring atom is a heteroatom such as, but not limited to, N, O, and S. Thus a heterocyclyl can be a cycloheteroalkyl, or a heteroaryl, or if polycyclic, any combination thereof.

Heteroaryl groups are heterocyclic aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure, which is a multiply-unsaturated cyclic system that contains 4n+2π electrons wherein n is an integer.

### Documents Cited

1. Takaishi, H.; Kimura, T.; Dalai, S.; Okada, Y.; D'Armiento, J. Joint diseases and matrix metalloproteinases: A role for MMP-13. Curr. Pharm. Biotechnol. 2008, 9, 47-54.
2. Neuhold., L. A.; Killar, L.; Zhao, W. G.; Sung, M. L. A.; Warner, L.; Kulik, J.; Turner, J.; Wu, W.; Billinghurst, C,; Meijers, T.; Poole, A. R.; Babij P.; DeGennaro, L. J. Postnatal expression in hyaline cartilage of constitutively active human collagenase-3 (MMP-13) induces osteoarthritis in mice. J. Clin. Invest. 2001, 107, 35-44.
3. Bau, B.; Gebhard, P. M.; Haag, J.; Knorr, T.; Bartnik, E.; Aigner, T. Relative messenger RNA expression profiling of collagenases and aggrecanases in human articular chondrocytes in vivo and in vitro. Arthritis and Rheumatism 2002, 46, 2648-2657.
4. Morrison, C.; Mancini, S.; Cipollone, J.; Kappelhoff, R.; Roskelley, C.; Overall, C. Microarray and Proteornic Analysis of Breast Cancer Cell and Osteoblast Co-cultures: Role of Osteoblast Matrix Metalloproteinase (MMP)-13 in Bone Metastasis. J. Biol. Chem. 2011, 286, 34271-34285,
5. Vandenbroucke, R. E.; Dejonckheere, E.; Van Hauwermeiren, F.; Lodens, S.; De Rycke, R.; Van Wonterghem, E.; Staes, A.; Gevaert, K.; López-Orin, C.; Libert, C. Matrix metalloproteinase 13 modulates intestinal epithelial barrier integrity in inflammatory diseases by activating TNF. EMBO Molecular Medicine 2013, 5, 1000-1016.
6. Zigrino, P.; Kuhn, I.; Bauerle, T.; Zamek, J.; Fox, J. W.; Neumann, S.; Licht, A.; Schorpp-Kistner, M.; Angel, P.; Mauch, C. Stromal Expression of MMP-13 is required for Melanoma. Invasion and Metastasis. J. Invest. Dermatol. 2009, 129, 2686-2693.
7. Lukacova, V.; Zhang, Y.; Mackov, M.; Baricic, P.; Raha, S.; Calvo, J. A.; Balaz, S. Similarity of Binding Sites of Human Matrix Metalloproteinases. J. Biol. Chem. 2004, 279, 14194-14200.
8. Lovejoy, B.; Welch, A. R.; Carr, S.; Luong, Broka, C.; Hendricks, R. T.; Campbell, J. A.; Walker, K. A. M.; Martin, R.; Van Wart, H.; Browner, M. F. Crystal structures of MMP-1 and -13 reveal the structural basis for selectivity of collagenase inhibitors, Nat. Struct. Biol. 1999, 6, 217-221.
9. Rothenberg, M. L.; Nelson, A. R.; Mande, K. R. New Drugs on the Horizon: Matrix Metalloproteinase Inhibitors. Stem Cells 1999, 17, 237-240.
10. Blagg, J. A.; Noe, M. C.; Wolf-Gouveia, L. A.; Reiter, L. A.; Laird, E. R.; Chang, S.-P. P.; Danley, D. E.; Downs, J. T.; Elliott, N. C.; Eskra, J. D.; Griffiths, R. J.; Hardink, J. R.; Haugeto, A. I.; Jones, C. S.; Liras, J. L.; Lopresti-Morrow, L. L.; Mitchell, P. G.; Pandit, J.; Robinson, R. P.; Subramanyam, C.; Vaughn-Bowser, M. L.; Yocum, S. A. Potent pyrimidinetrione-based inhibitors of MMP-13 with enhanced selectivity over MMP-14. Bioorg. Med. Chem. Lett. 2005, 15, 1807-1810.
11. Mengshol, J. A.; Mix, K. S.; Brinckerhoff, C. E. Matrix metalloproteinases as therapeutic targets in arthritic diseases: Bull's-eye or missing the mark? Arthritis and Rheumatism 2002, 46, 13-20.
12. Burrage, K. S.; Mix, K. S.; Brinckerhoff, C. I. Matrix metalloproteinases: Role in arthritis. Front. Biosci. 2006, 11, 529-543.
13. Fingleton, B. MMPs as therapeutic targets—Still a viable option? Seminars in Cell & Developmental Biology 2008, 19, 61-68.
14. Nara, H.; Kaieda., A.; Sato, K.; Naito, I.; Mototani, H.; Oki, H.; Yamamoto, Y.; Kuno, H.; Santou, T.; Kanzaki, N.; Terauchi, J.; Uchikawa, O.; Kori, M. Discovery of Novel, Highly Potent, and Selective Matrix Metalloproteinase (MMP)-13 Inhibitors with a 1,2,4-Triazol-3-yl Moiety as a Zinc Binding Group Using a Structure-Based. Design Approach. J. Med. Chem. 2017, 60, 608-626.
15. Dormán, G.; Cseh, S.; Hajdú, I; Barna, L.; Kónya, D.; Kupai, K.; Kovács, L.; Ferdinandy, P. Matrix Metalloproteinase Inhibitors. Drugs 2010 949-964.
16. Nara, H.; Sato, K.; Naito, T.; Mototani, H.; Old, H.; Yamamoto, Y.; Kuno, H.; Santou, T.; Kanzald, N.; Terauchi, J.; Uchikawa, O.; Kori, M. Discovery of Novel, Highly Potent, and Selective Quinazoline-2-carboxamide-Based Matrix Metalloproteinase (MMP)-13 Inhibitors without a Zinc Binding Group Using a Structure-Based Design Approach. J. Med. Chem. 2014, 57, 8886-8902.
17. liege, C.; Bao, B.; Bluhm, H.; Boer, J.; Gallagher, B. M.; Korniski, B.; Powers, T. S.; Steeneck, C.; Taveras, A. G.; Baragi, V. M. Discovery and Evaluation of a Non-Zn Chelating, Selective Matrix Metalloproteinase 13 (MMP-13) Inhibitor for Potential Intra-articular Treatment of Osteoarthritis. J. Med. Chem. 2011, 55, 709-716.
18. Johnson, A. R.; Pavlovsky, A. G.; Ortwine, D. F.; Prior, F.; Man, C. F.; Bornemeier, D. A.; Banotai, C. A.; Mueller, W. T.; Mcconnell, P.; Yan, C.; Baragi, V.; Lesch, C.; Roark, W. H.; Wilson, M.; Datta, K.; Guzman, R.; Han, H. K.; Dyer, R. D. Discovery and characterization of a novel inhibitor of matrix metalloprotease-13 that reduces cartilage damage in vivo without joint fibroplasia side effects. J. Biol. Chem. 2007, 282, 27781-27791.
19. Nara, H.; Sato, K.; Kaieda, A.; Oki, H.; Kuno, H.; Santou, T.; Kanzaki, N.; Terauchi, J.; Uchikawa, O.; Kori, M. Design, synthesis, and biological activity of novel, potent, and highly selective fused pyrimidine-2-carboxamide-4-one-based matrix metalloproteinase (MMP)-13 zinc-binding inhibitors. Bioorg. Med. Chem. 2016, 24, 6149-6165.
20. Thiel, K. A. Structure-aided drug design's next generation. Nat. Biotechnol. 2004, 22, 513-519.
21. Sliwoski, G.; Kothiwale, S.; Meiler, J.; Lowe, E. W. Computational Methods in Drug Discovery. Pharmacol. Rev. 2014, 66, 334-395.
22. Kuntz, I. D. Structure-Based. Strategies for Drug Design and Discovery. Science 1992, 257, 1078-1082.
23. Spicer, T. P.; Jiang, J. W.; Taylor, A. B.; Choi, J. Y.; Hart, J.; Roush, W. R.; Fields, G. B.; Hodder, P. S.; Minong, D. Characterization of Selective Exosite-Binding Inhibitors of Matrix Metalloproteinase 13 That Prevent Articular Cartilage Degradation in Vitro. J. Med. Chem. 2014, 57, 9598-9611.
24. Berman, H. M.; Westbrook, J.; Feng, Z.; Gilliland, G.; Bhat, T. N.; Weissig, H.; Shindyalov, I. N.; Bourne, P. E. The Protein Data Bank, Nucleic Acids Research 2000, 28, 235-242.
25. Roth J.; Minond, D.; Darout, E.; Liu, Q.; Lauer, J.; Hodder, P.; Fields, G. B.; Roush, W. R. Identification of novel, exosite-binding matrix metalloproteinase-13 inhibitor scaffolds. Bioorg. Med. Chem. Lett. 2011, 21, 7180-7184.
26. Lauer-Fields, J. L.; Fields, G. B. Triple-helical peptide analysis of collagenolytic protease activity. Biol. Chem. 2002, 383, 1095-1105.
27. Minond, D.; Lauer-Fields, J. L.; Cudic, M.; Overall, C. M.; Pei, D. Q.; Brew, K.; Visse, R.; Nagase, H.; Fields, G. B. The roles of substrate thermal stability and P-2 and P-1′ subsite identity on matrix metalloproteinase triple-helical peptidase activity and collagen specificity. J. Biol. Chem. 2006, 281, 38302-38313.
28. Parks, W. C.; Wilson, C. L.; Lopez-Boado, Y. S. Matrix metalloproteinases as modulators of inflammation and innate immunity. Nat. Rev. Immunol 2004, 4, 617-629.
29. Bigg, H. F.; Rowan, A. D.; Barker, M. D.; Cawston, T. E. Activity of matrix metalloproteinase-9 against native collagen types I and. III. FEBS Journal 2007, 274, 1246-1255.
30. Fields, G. B. Interstitial Collagen Catabolism. J. Biol. Chem. 2013, 288, 8785-8793.
31. Lauer-Fields, J. L.; Broder, T.; Sritharan, T.; Chung, L.; Nagase, H.; Fields, G. B. Kinetic Analysis of Matrix Metalloproteinase Activity Using Fluorogenic Triple-Helical Substrates. Biochem 2001, 40, 5795-5803.
32. Chung, L.; Dinakarpandian, D.; Yoshida, N.; Lauer-Fields, J. L.; Fields, G. B.; Visse, R.; Nagase, H. Collagenase unwinds triple-helical collagen prior to peptide bond hydrolysis. EMBO 2004, 23, 3020-3030.
33. Neumann, U.; Kubota, H.; Frei, K.; Ganu, V.; Leppert, D. Characterization of Mca-Lys-Pro-Leu-Gly-Leu-Dpa-Ala-Arg-NH2, a fluorogenic substrate with increased specificity constants for collagenases and tumor necrosis factor converting enzyme. Anal. Biochem. 2004, 328, 166-173.

All patents and publications referred to herein are incorporated by reference herein to the same extent as if each individual publication was specifically and individually indicated to be incorporated by reference in its entirety.

### EXAMPLES

### Abbreviations

MMP, matrix metalloproteinase; RMSD, root mean square deviation; THP, triple helical peptide; FAM-fTHP, fluorescein amidite-fluorescence resonance energy transfer triple-helical peptide, OA, osteoarthritis; PDB, protein data bank; ACE, angiotensin-converting enzyme; ADAM, a disintegrin and metalloproteinase; BACE, beta-secretase; IDE, insulin degrading enzyme; TACE, tumor necrosis factor-α-converting enzyme; uPA, urokinase-type plasminogen activator.

General experimental details: All non-aqueous reactions were carried out under a positive pressure of argon using oven-dried (140° C.) or flame-dried glassware (under vacuum). Dichloromethane, diethyl ether, N,N-dimethylformamide, toluene and tetrahydrofuran were dried by being passed through a column of desiccant (activated A-1 alumina). Triethylamine was distilled from calcium hydride under an argon atmosphere prior to use. All other commercially available reagents were used without further purification. Reactions were either monitored by thin layer chromatography or analytical LC-MS. Thin layer chromatography was performed on Kieselgel 60 F254 glass plates pre-coated with a 0.25 mm thickness of silica gel. TLC plates were visualized with UV light and/or by staining with Hanessian solution [H₂SO₄ (conc., 22 mL), phosphormolybdic acid (20 g), Ce(SO₄)₂ (0.5 g), 378 mL H₂O)].

Column chromatography was performed on a Biotage Isolera automated flash system. Compound was loaded onto pre-filled cartridges filled with KP-Sil 50 μm irregular silica.

NMR spectra were recorded on a 400 MHz spectrometer and measured in CDCl₃, MeOD or DMSO (CHCl₃: ¹H, δ=7.26, ¹³C, δ=77.16, MeOH: ¹H, δ=3.31, ¹³C, δ=49.00, DMSO: ¹H, δ=2.50, ¹³C, δ=39.50). All ¹H and ¹³C shifts are given in ppm and coupling constants J are given in Hz.

High-resolution mass spectra were recorded on a spectrometer (ESI) at the University of Illinois Urbana-Champaign Mass Spectrometry Laboratory.

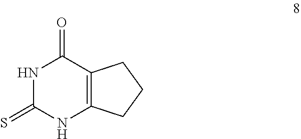 [SMILES: O=c1[nH]c(=S)[nH]c2c1CCC2]

DRU (11.5 mL, 77.33 mmol, L1 eq) was added to a suspension of methyl-2-cyclopentanone-1-carboxylate (10 g, 70.3 mmol, 1.0 eq) and thiourea (8.03 g, 105.45 mmol, 1.5 eq) in 70 mL CH₃CN and the mixture was stirred at 80° C. for 16 h. The reaction mixture was cooled to 0° C. while a white solid precipitated. The solid product was filtered, washed with 2M HCl (2×30 mL) and water (2×30 mL) and was dried under vacuum to give 8 (7.54 g, 64%) as a white powder.

¹H NMR (400 MHz, DMSO) δ=12.59; (s, 1H), 12.21; (s, 1H), 2.76-2.63; (m, 2H), 2.56-2.44; (m, 2H), 2.05-1.87; (m, 2H).

¹³C NMR (101 MHz, DMSO) δ 175.54, 159.51, 156.45, 115.54, 30.98, 26.60, 20.78.

MS (ESI) for C₇H₈N₂OS [M+H]⁺ 169.10.

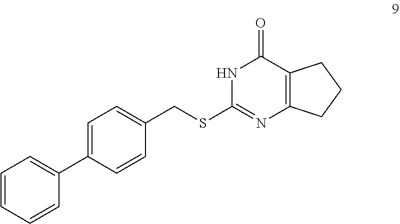 [SMILES: O=c1[nH]c(SCc2ccc(-c3ccccc3)cc2)nc2c1CCC2]

A suspension of 8 (1.22 g, 7.28 mmol, 1.2 eq) and triethylamine (1.0 mL, 7.28 mmol, 1.2 eq) in 12 mL DMF was stirred for 20 min at room temperature before 4-bromomethylbiphenyl (1.5 g, 6.07 mmol, 1.0 eq) was added and the reaction mixture was stirred for 16 h at room temperature. The solids were filtered, washed with small amounts of water, methanol and diethyl ether and the product was dried under vacuum to give 9 (1.9 g, 94%) as a white solid.

¹H NMR (400 MHz, DMSO) δ=12.56; (bs, 1H), 7.71-7.56; (m, 4H), 7.54-7.41; (m, 4H), 7.40-7.31; (m, 1H), 4.43; (s, 2H), 2.85-2.73; (m, 2H), 2.63-2.56; (m, 2H), 2.03-1.90; (m, 2H).

¹³C NMR (101 MHz, DMSO) δ 139.71, 139.14, 136.46, 129.65, 128.90, 127.43, 126.73, 126.58, 34.28, 33.23, 26.71, 20.56.

MS (ESI) for C₂₀H₁₈N₂OS [M+H]⁺ 335.11.

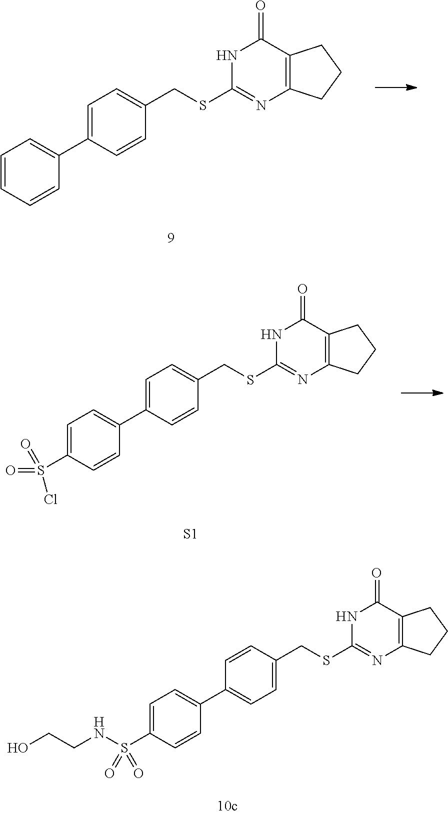 [SMILES: O=c1[nH]c(SCc2ccc(-c3ccccc3)cc2)nc2c1CCC2 ; O=c1[nH]c(SCc2ccc(-c3ccc(S(=O)(=O)Cl)cc3)cc2)nc2c1CCC2 ; "S1" ; O=c1[nH]c(SCc2ccc(-c3ccc(S(=O)(=O)NCCO)cc3)cc2)nc2c1CCC2]

Chlorosulfonic acid (3M in DCM, 4 mL, 11.96 mmol, 20.0 eq) vas added to 9 (200 mg, 0.598 mmol, 1.0 eq) at 0° C., the cooling bath was removed and the deep blue solution was stirred for 16 hours at room temperature. The reaction was quenched by pouring it onto a mixture of ice-water (ca. 20 mL) and ethyl acetate (ca. 10 mL), further 10 mL of THF were added to dissolve the white solids formed during the quench. The phases were separated and the organic layer was dried over Na2SO4, filtered and the solvent was removed under reduced pressure to provide the crude sulfonyl chloride (S1, 200 mg, 77%) as a white solid.

The sulfonyl chloride (S1, 106 mg, 0.245 mmol, 1.0 eq) was dissolved in 2.2 mL THF and 2-ethanolamine (22 μL, 0.368 mmol, 1.5 eq) followed by triethylamine (68 μL, 0.49 mmol, 2.0 eq) were added at room temperature. The reaction mixture was stirred for 16 h, the solvent was removed under reduced pressure and the product was purified by flash chromatography (0-10% methanol linear gradient in DCM) to provide 10c (30 mg, 27%) as a white solid.

¹H NMR (400 MHz, DMSO) δ=12,51 (bs, 1H), 7.87; (s, 4H), 7.74-7.63; (m, 3H), 7.54; (J=8.3 Hz, 2H), 4.80-4.64; (m, 1H), 4.45; (s, 2H), 3.45-3.30; (m, 4H), 2.81-2.74; (m, 21), 2.59; (J=7.4 Hz, 2H), 2.02-1.91; (m, 2H).

¹³C NMR (101 MHz, DMSO) δ 168.74, 160.69, 159.53, 143.36, 139.33, 137.75, 137.48, 129.82, 127.23, 127.15, 127.11, 119.41, 59.92, 45.11, 40.13, 39.92, 39.71, 39.50, 39.29, 39.08, 38,88, 34.28, 33.17, 26.72, 20.57.

IR (thin film) v 3374, 2923, 1655, 1316, 1158, 1050 cm⁻¹.

HRMS (ESI) calcd. for C₂₂H₂₃N₃O₄S₂[M+H]⁺ 458.1130; found 458.1215

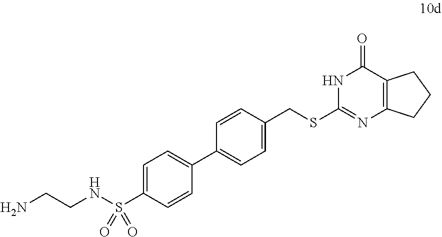 [SMILES: NCCNS(=O)(=O)c1ccc(-c2ccc(CSc3nc4c(c(=O)[nH]3)CCC4)cc2)cc1]

Chlorosulfonic acid (3M in L)CM, 4 mL, 11.96 mmol, 20.0 eq) was added to 9 (200 mg, 0.598 mmol, 1.0 eq) at 0° C., the cooling bath was removed and the deep blue solution was stirred for 16 h at room temperature. The reaction was quenched by pouring it onto a mixture of ice-water (ca. 20 mL) and ethyl acetate (ca. 10 mL), further 10 mL of THF were added to completely dissolve all the solids. The phases were separated and the organic layer was dried over Na₂SO₄, filtered and the solvent was removed under reduced pressure to provide the crude sulfonyl chloride (S1, 200 mg, 77%) as a white solid.

The sulfonyl chloride (S1, 106 mg, 0,245 mmol, 1.0 eq) was dissolved in 2.2 mL THF and mono-Boc-protected ethylenediamine (59 mg, 0.368 mmol, 1.5 eq) followed by tritehylamine (68 μL, 0.49 mmol, 2.0 eq) were added at room temperature. The reaction mixture was stirred for 16 h, the solvent was removed under reduced pressure and the product was purified by flash chromatography (0-10% methanol linear gradient in DCM) to provide Boc-protected 10d (41 mg, 30%) as a white solid. ¹H NMR (400 MHz, DMSO) δ=12.58; (s, 1H), 7.93-7.81; (m, 4H), 7.76-7.67; (m, 3H), 7.58-7.50; (m, 2H), 6.81; (t, J=5.8, 1H), 4.45; (s, 2H), 3.03-2.93; (m, 2H), 2.83-2.75; (m, 4H), 2.64-2.57; (m, 2H), 2.03-1.92; (m, 2H), 1.34; (s, 9H).

Boc-protected 10d (41 mg, 0.074 mmol) was suspended in 1 mL HCl in dioxane (4M). The reaction mixture was stirred for 90 min at room temperature and concentrated under reduced pressure. The crude product was triturated with diethyl ether (1×1.5 mL) followed by diethyl ether/methanol=20/1 (2×1.5 mL) and dried under vacuum to give 10d (29 mg, 85%) as a slightly yellow solid.

¹H NMR (400 MHz, DMSO) δ=8.34-8.12; (m, 4H), 7.89; (s, 4H), 7.69; (d, J=8.3, 2H), 7.54; (d, J=8.3, 2H), 4.45; (s, 2H), 3.08-299; (m, 214), 2.91-2.82; (m, 2H), 2.81-2.74; (m, 2H), 2.58; (t, J==7.4, 2H), 2.01-1.89; (m, 2H).

¹³C NMR (101 MHz, DMSO) δ 168.50, 161.15, 160.21, 143.76, 138.48, 137.88, 137.44, 129.92, 127.44, 127.40, 127.21, 119.41, 40.08, 38.57, 34.13, 33.25, 26.75, 20.67.

IR. (thin film) v 3376, 1645, 1566, 1046, 990 cm⁻¹.

HRMS (ESI) calcd. for C₂₂H₂₄N₄O₃S₂ [M+H]⁺ 457.1290; found 457.1363.

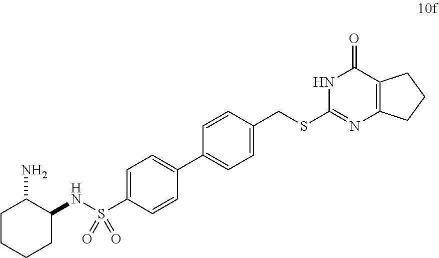

The sulfonyl chloride (S1) synthesized as described above for 10e.

The sulfonyl chloride (131 mg, 0.303 mmol, 1.0 eq) was dissolved in 2.75 mL. THF and (±1,2-trans-cyclohexanediamine (52 mg, 0.455 mmol, 1.5 eq) followed by triethylamine (84 ∛L, 0.606 mmol, 2.0 eq) were added at room temperature. The reaction mixture was stirred for 16 h, the precipitate was filtered and washed with small amounts of THF and diethyl ether. The crude product was further purified by preparative HPLC (linear gradient 10-100% acetonitrile/MeOH=1:1, 0.1% TFA, 10 min). Lyophilization gave 26 mg (28%) of 10f as a slightly yellow powder.

¹H NMR (400 MHz, DMSO) δ=12.57 (bs, 1H), 8.03; (d, J=8.8 Hz, 1H), 7.97-7.86; (m, 6H), 7.75-7.70; (m, 2H), 7.58-7.52; (m, 2H), 4.45; (s, 2H), 3.06-2.94; (m, 1H), 2.85-2.71; (m, 3H), 2.67-2.55; (m, 2H), 2.03-1.92; (m, 3H), 1.63-1.43; (m, 2H), 1.41-1.26; (m, 1H), 1.25-1.05; (m, 3H), 1.05-0.86; (m, 1H).

¹³C NMR (101 MHz, DMSO) δ 158.26, 157.95, 143.58, 140.31, 137.99, 137.22, 129.89, 127.34, 127.14, 118.71, 54.81, 53.43, 34,27, 33.18, 30.46, 29.09, 26.75, 23.87, 22.97, 20.60.

TR (thin film) v 29:33, 2861, 1651, 1531, 1427, 1315, 1152, 1056 cm⁻¹.

HRMS (ESI) calcd. for C₂₆H₃₀N₄O₃S₂ [M+H]⁺ 511,1759; found 511.1818.

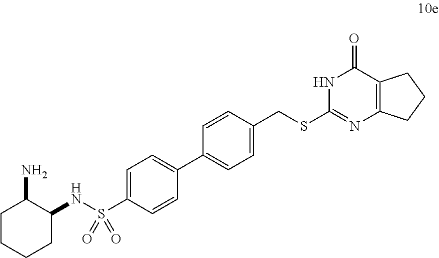

Compound 10e was prepared as described for 10c using (±)-1,2-cis-cyclohexanediamine as the coupling partner for the sulfonyl chloride S1. 10e (23 mg) was isolated in 24% yield.

¹H NMR (400 MHz, DMSO) δ=12.57; (bs, 1H), 7.97-7.89; (m, 5H), 7.84-7.77; (m, 2H), 7.72; (d, J=8.4 Hz, 2H), 7.55; (d, J=8.4 Hz, 2H), 4.45; (s, 2H), 3.45-3.39; (m, 1H), 3.21-3.10; (m, 1H), 2.82-2.73; (m, 2H), 2.59; (t, J=7.3 Hz, 2H), 1.96; (p, J=7.3 Hz, 2H), 1.68-1.47; (m, 4H), 1.33-1.08; (m, 4H).

HRMS (EST) calcd. for C₂₆H₃₀N₄O₃S₂ [M+H]⁺ 511.1759; found 511.1823.

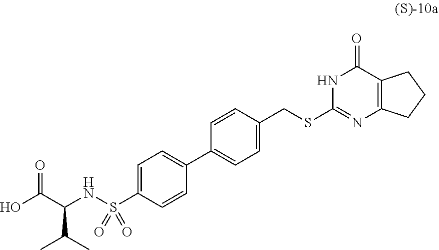 [SMILES: CC(C)[C@H](NS(=O)(=O)c1ccc(-c2ccc(CSc3nc4c(c(=O)[nH]3)CCC4)cc2)cc1)C(=O)O]

The sulfonyl chloride (S1) was synthesized as described above for 10c.

A solution of L-valine methyl ester⋅HCl (150 mg, 0.897 mmol, 1.5 eq) in 0.9 mL THF and 0.9 ml. water was added to the sulfonyl chloride (S1, 259 mg, 0.598 mmol, 1.0 eq) and triethylamine (249 μL, 1.794 mmol, 3.0 eq) was added at room temperature. The reaction mixture was stirred for 24 h before it was diluted with ethyl acetate (10 mL) and water (10 mL). The phases were separated and the aqueous phase was extracted with ethyl acetate (3×10 mL). The combined organic extracts were dried over Na₂SO₄, filtered and the solvent was removed under reduced pressure. The crude methyl ester was purified by flash chromatography (0-10% methanol linear gradient in DCM). The product was further purified by trituration with diethyl ether (1×1.5 mL) followed by diethyl etherlinethanol=20/1 (2×1.5 mL) and dried under vacuum to give the corresponding valine methyl ester (67 mg, 21%) as a slightly yellow solid. ¹H NMR (400 MHz, DMSO) δ=12.40 (s, 1H), 8.31; (J=9.4 Hz, 1H), 7.89-7.82; (m, 2H), 7.82-7.76; (m, 2H), 7.72-7.64; (m, 2H), 7.57-7.50; (m, 2H), 4.44; (s, 2H), 3.56; (dd, J=9.3, 7.1 Hz, 1H), 3.32; (s, 3H), 2.83-2.72; (m, 2H), 2.59; (t, J=7.4 Hz, 2H), 1.96; (p, J=7.4 Hz, 2H), 0.83; (J=6.7 Hz, 3H), 0.79; (d, J=6.7 Hz, 3H). MS (ESI) for C₂₆H₂₉N₃O₅S₂ [M+H]⁺ 528.20.

Lithium hydroxide (7 mg, 0.29 mmol, 3.0 eq) was added to a solution of the methyl ester in 2 mL of a 1:1 mixture of THF and water. The reaction mixture was heated to 60° C. and stirred for 16 h before it was acidified with 1M HCl (pH˜3). Filtration of the precipitated product gave (S)-10a (29 mg) in 58% yield.

¹H NMR (400 MHz, DMSO) δ=12.57; (s, 2H), 8.07; (d, J=9.3 Hz, 1H), 7.83; (s, 4H), 7.69; (d, J=8.2 Hz, 2H), 7.53; (d, J=8.2 Hz, 2H), 4.44; (s, 2H), 3.55; (dd, 9.3, 5.9 Hz, 1H), 2.78; (J=7.7 Hz, 2H), 2.59; (t, J=7.7 Hz, 2H), 2.06-1.86; (m, 311 0.83; (d, J=6.7 Hz, 3H), 0.80; (d, J=6.7 Hz, 3H).

¹³C NMR (101 MHz, DMSO) δ 172.18, 168.77, 160,77, 159.92, 143.10, 139.96, 137.73, 137.38, 129.82, 127.19, 127.04, 126.84, 119.52, 61.26, 40.13, 34.28, 33.15, 30.40, 26.71, 20.57, 19.02, 17.86.

IR (thin film) v 2965, 1647, 1548, 1192, 1166, 1096 cm⁻¹.

HRMS (ESI) calcd. for C₂₅H₂₇N₃O₅S₂ [M+H]⁺ 514.1392; found 514.1476.

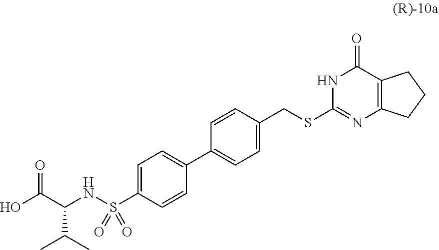 [SMILES: CC(C)[C@@H](NS(=O)(=O)c1ccc(-c2ccc(CSc3nc4c(c(=O)[nH]3)CCC4)cc2)cc1)C(=O)O]

Compound (R)-10a was prepared as described for (S)-10a using D-valine methyl ester⋅HCl as the coupling partner for the sulfonyl chloride (S1). (R)-10a (44 mg) was isolated in 62% yield.

¹H NMR (400 MHz, DMSO) δ=12.57; (s, 2H), 8.07; (dJ=9.3 Hz, 1H), 7.83; (s, 4H), 7.69; (d, J=8.2 Hz, 2H), 7.53; (d, J=8.2 Hz, 2H), 4.44; (s, 2H), 3.55; (dd, J=9.3, 5.9 Hz, 1H), 2.78; (t, J=7.7 Hz, 2H), 2.59; (t, J=7.7 Hz, 2H), 2.06-1.86; (m, 3H), 0.83; (d, J=6.7 Hz, 3H), 0.80; (d, =6.7 Hz, 3H).

¹³C NMR (101 MHz, DMSO) δ 172.18, 168.77, 160.77, 159.98, 143.10, 139.96, 137.73, 137.38, 129.82, 127.19, 127.04, 126.84, 119.52, 61.26, 40.13, 34.28, 33.15, 30.40, 26.71, 20.57, 19,02, 17.86.

IR (thin film) v 2965, 1647, 1548, 1192, 1166, 1096 cm⁻¹.

HRMS (ESI) calcd. for C₂₅H₂₇N₃O₅S₂ [M+H]⁺ 514.1392; found 514.1465.

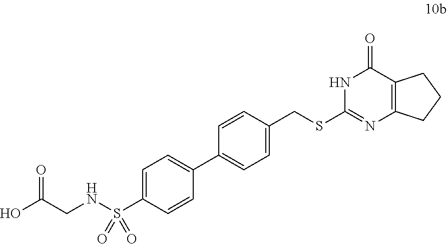 [SMILES: O=C(O)CNS(=O)(=O)c1ccc(-c2ccc(CSc3nc4c(c(=O)[nH]3)CCC4)cc2)cc1]

Compound 10b was prepared as described for (S)-10a using glycine-tertbutyl-ester (150 mg, 0.897 mmol) as the coupling partner for the sulfonyl chloride (S1; yield (tert-butyl ester): 70 mg, 45%).

Ester hydrolysis: The tert-butyl ester (65 mg, 0.123 mmol, 1.0 eq) was dissolved in 2 mL of a 1:1 mixture of CH₂Cl₂ and trifluoroacetic acid. The reaction mixture was stirred for 2 h, concentrated under reduced pressure and the crude product was purified by trituration with diethyl ether (1×1.5 mL) followed by diethyl ether/methanol-20/1 (2×1.5 mL) and dried under vacuum to give 10b (26 mg, 45%) as a slightly yellow solid.

¹H NMR (400 MHz, DMSO) δ=12.61; (s, 2H), 8.09; (t, J=6.1 Hz, 1H), 7.85; (s, 4H), 7.69; (d, J=8.3 Hz, 1H), 7.54; (J=8.3 Hz, 1H), 4.44; (s, 2H), 3.62; (d, J=5.4 Hz, 2H), 2.78; (t, J=7.7 Hz, 2H), 2.59; (t, J=7.4 Hz, 2H), 2.04-1.88; (2H).

¹³C NMR (101 MHz, DMSO) δ 170.24, 143.38, 139.46, 137.75, 137.44, 129.81, 127.14, 127.11, 127.09, 43.80, 34.27, 33.15, 26.71, 20.56.

IR (thin film) v 2939, 1650, 1548, 1192, 1159, 1096 cm⁻¹.

HRMS (ESI) calcd. for C₂₂H₂₁N₃O₅S₂ [M+H]⁺ 472.0923; found 472.1000.

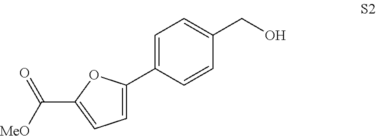 [SMILES: COC(=O)c1ccc(-c2ccc(CO)cc2)o1]

To a solution of methyl-2-bromo-5-furanocarboxylate (1.0 g, 4.88 mmol, 1.0 eq) in 150 mL dioxane was added Pd(PPh₃)₄ (76 mg, 0.239 mmol, 0.05 eq) and the resulting yellow solution was stirred for 15 min at room temperature. 4-Hydroxymethylbenzeneboronic acid (741 mg, 4.88 mmol, 1.0 eq) dissolved in 45 mL, water followed by K₂CO₃ (810 mg, 5.86 mmol, 1.2 eq) were added and the reaction mixture was stirred at 60° C. for 16 h. After cooling to room temperature most of the dioxane was removed under reduced pressure. The residue was diluted with water (ca. 50 mL) and the product was extracted with ethyl acetate (3×50 mL). The combined organic extracts were dried over Na₂SO₄, filtered and the solvent was removed under reduced pressure. The crude product was purified by flash chromatography (6-37% ethyl acetate gradient in hexanes) providing S2 (987 mg, 87%) as a slightly yellow solid.

¹H NMR (400 MHz, CDCl₃) δ=7.75; (d, J=8.1 Hz, 2H), 7.39; (d, J=8.1 Hz, 2H), 7.23; (d, J=3.6 Hz, 1H), 6.72; (d, J=3.6 Hz, 1H), 4.71; (s, 2H), 3.90; (s, 3H).

¹³C NMR (101 MHz, CDCl₃) δ 159.40, 157,51, 143.56, 141.86, 128.82, 127.39, 125.10, 120.25, 106.97, 64.94, 52.03.

MS (ESI) for C₁₃H₁₂O₄ [M+H]⁺ 233.09.

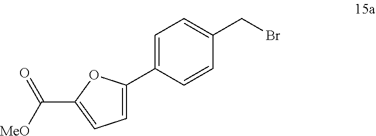 [SMILES: COC(=O)c1ccc(-c2ccc(CBr)cc2)o1]

To a stirring solution of benzylic alcohol S2 (980 mg, 4.22 mmol, 1.0 eq) and CBr4 (1.82 g, 5.49 mmol, 1.3 eq) in 14 mL methylene chloride was added PPh₃ (1.44 g, 5.49 mmol, 1.3 eq) at 0° C. The reaction mixture was stirred for 1 h at 0° C., quenched with water and the product was extracted with methylene chloride (3×15 mL). The combined organic extracts were dried over Na₂SO₄, filtered and the solvent was removed under reduced pressure. The crude product was purified by flash chromatography (4-34% ethyl acetate gradient in hexanes) giving 15a (1.14 g, 91%) as a slightly yellow solid.

¹H NMR (400 MHz, CDCl₃) δ=7.73; (d, J=8.3 Hz, 1H), 7.42; (d, J=8.3 Hz, 2H), 7.23; (d, J=3.6 Hz, 1H), 6.74; (d, J=3.4 Hz, 1H), 4.49; (s, 2H), 3.91; (s, 2H).

¹³C NMR (101 MHz, CDCl₃) δ 159.20, 156,92, 143.87, 138.51, 129.65, 129.54, 125.26, 120.11, 107.49, 52.01, 33.08.

MS (ESI) for C₁₃H₁₁BrO₃ [M+H]⁺ 295.05.

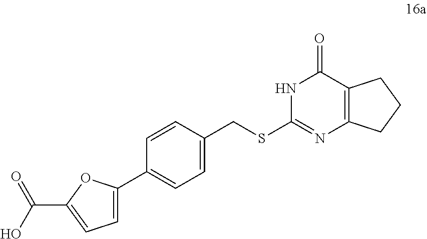 [SMILES: O=C(O)c1ccc(-c2ccc(CSc3nc4c(c(=O)[nH]3)CCC4)cc2)o1]

A suspension of 8 (476 mg, 2.83 mmol, 1.0 eq) and triethylamine (470 μL, 3.39 mmol, 1.2 eq) in 7 mL DMF was stirred for 20 min at room temperature before 15a (1 g, 3.39 mmol, 1.2 eq) was added and the reaction mixture was stirred for 16 h at room temperature. The solids were filtered, washed with small amounts of water, methanol and diethyl ether and the product was dried under vacuum to give the corresponding methyl ester (1.16 g, 89%) as a white solid. ¹H NMR (400 MHz, DMSO) δ=12.55; (bs, 1H), 7.76; (d, J=8.4, 2H), 7.52; (d, J=8.4, 2H), 7.41; (d, J=3.7, 1H), 7.15; (d, J=3.7, 1H), 4.42; (s, 2H), 3.83; (s, 3H), 2.81-2.73; (m, 2H), 2.64-2.54; (m, 2H), 2.07-1.87; (m, 2H). MS (ESI) for C₂₀H₁₈N₂O₄S [M+H]⁺, 383.10.

An aqueous solution of NaOH (1 M, 4.96 mL, 4.96 mmol, 3.2 eq) was added to a suspension of the methyl ester from above (594 mg, 1.55 mmol, 1.0 eq) in 16 mL of a 2:1 mixture of THF and methanol and the reaction mixture was heated to 60° C. for 2 h. After cooling to room temperature the mixture was diluted with water (ca. 2 mL) and acidified with 1 M HCl (pH˜2, ca. 5 mL), The precipitated product was filtered and washed with water providing 16a (554 mg, 97%) as a white solid.

¹H NMR (400 MHz, DMSO) δ=13.07; (bs, 1H), 12.59; (bs, 1H), 7.75; (d, J=8.4 Hz, 2H), 7.52; (d, J=8.4 Hz, 2H), 7.31; (d, J=3.6 Hz, 1H), 7.12; (d, J=3.6 Hz, 1H), 4.42; (s, 2H), 2.84-2.73; (m, 2H), 2.63-2.55; (m, 2H), 2.03-1.89; (m, 2H).

¹³C NMR (101 MHz, DMSO) δ 168.76, 160.72, 159.89, 159.27, 156.05, 144.13, 138.30, 129.84, 128.14, 124.46, 119.90, 119.44, 107.96, 34.28, 33.30, 26.74, 20.58.

MS (ESI) for C₁₉H₁₆N₂O₄S [M+H]⁺ 368.17.

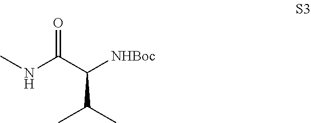 [SMILES: CNC(=O)[C@@H](NC(=O)OC(C)(C)C)C(C)C]

To a solution of Boc-protected L-valine (3.0 g, 13.8 mmol, 1.0 eq), EDCI-HCl (3.17 g, 16.56 mmol, 1.2 eq), and DMAP (100 mg, 0.82 mmol, 0.05 eq) in 138 mL methylene chloride was added methylamine (2 M in THF, 8.25 mL, 16.56 mmol, 1.2 eq). The reaction mixture was stirred for 18 h at room temperature before it was transferred in a separatory funnel and washed with 1 M HCl (2×100 mL), aqueous saturated solution of NaHCO₃ (2×100 mL) and brine (1×100 mL). The organic extract was dried over Na₂SO₄ and concentrated under reduced pressure to provide S3 (2.8 g, 88%) as a yellow oil, which was used for the next step without any further purification.

¹H NMR (400 MHz, CDCl₃) δ=6.05; (s, 1H), 5.06; (bd, J=9.0 Hz, 1H), 3.87; (dd, Hz, 6.3, 1H), 2.82; (d, J=4.9 Hz, 3H), 2.18-2.04; (m, 1H), 1.44; (s, 9H), 0.94; (d, J=7.0 Hz, 3H), 0.91; (d, J=7.0 Hz, 3H).

These spectral characteristics are identical to those previously reported.¹

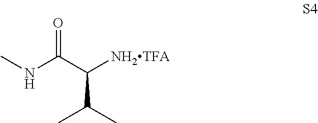

Compound S3 (1.0 g, 4.34 mmol, 1.0 eq) was dissolved in a 1:1 mixture of CH₂Cl₂ and trifluoroacetic acid (44 mL) and stirred for 90 min. The reaction mixture was concentrated under reduced pressure, the remaining yellow oil was re-dissolved in chloroform (ca. 10 mL) and the solvent was removed again in vacuo. This last step was repeated three times to eliminate all traces of trifluoroacetic acid and S4 (TFA-salt, 1.02 g) was isolated in 99% yield. The NMR of the crude material showed clean product, which was used without any further purification for the next step.

¹H NMR (400 MHz, MeOD) δ=3.60; (d, J=6.1 Hz, 1H), 2.82; (s, 3H), 2.23-2.10; (m, 1H), 1.05; (d, J=6.9 Hz, 6H).

These spectral characteristics were identical to those previously reported.¹

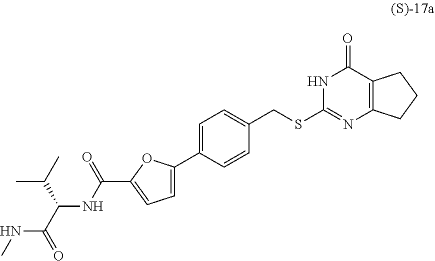 [SMILES: CNC(=O)[C@@H](NC(=O)c1ccc(-c2ccc(CSc3nc4c(c(=O)[nH]3)CCC4)cc2)o1)C(C)C]

To a solution of 16a (460 mg, 1.25 mmol, 1.0 eq), S4 (366 mg, 1.50 mmol, 1.2 eq) and HOBt (186 mg, 1.375 mmol, L1 eq) was added triethylamine (487 μL, 2.75 mmol, 2.2 eq). After stirring for 5 min at room temperature EDCI-HCl (264 mg, 1.38 mmol, 1.1 eq) was added and the clear solution was stirred for 6 h. The reaction mixture was diluted with ethyl acetate and washed with 0.1 M HCl (2×20 mL), sat. NaHCO₃ (1×20 mL) and brine (1×20 mL) The organic phase was dried over Na₂SO₄, filtered and the solvent was removed under reduced pressure. The crude product was purified by flash chromatography (0-10% methanol linear gradient in DCM) providing (S)-17a (467 mg, 79%) as a white solid.

¹H NMR (400 MHz, DMSO) δ=12.55; (s, 1H), 8.27; (d, J=8.9 Hz, 1H), 8.12-8.05; (m, 1H), 7.86; (d, J=8.4 Hz, 2H), 7.51; (d, J=8.4 Hz, 2H), 7.28; (d, J=3.6 Hz, 1H), 7.07; (d, J=3.6 Hz, 1H), 4.41; (s, 2H), 4.21; (t, J=8.7 Hz, 1H), 2.85-2.72; (m, 2H), 2.64-2.54; (m, 5H), 2.19-2.05; (m, 1H), 2.03-1.87; (m, 2H), 0.89; (t, J=6.9 Hz, 6H).

HRMS (ESI) calcd. for C₂₅H₂₈N₄O₄S [M+H]⁺ 481.1831; found 481.1902.

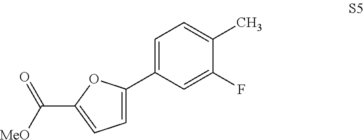 [SMILES: COC(=O)c1ccc(-c2ccc(C)c(F)c2)o1]

To a solution of methyl-2-bromo-5-furanocarboxylate (1.11 g, 5,42 mmol, 1.0 eq) in 22 mL toluene were added 3-fluoro-4-methylbenzeneboronic acid (1.0 g, 6.50 mmol, 1.2 eq) in 1.9 mL methanol followed by Pd(PPh₃)₄ (220 mg, 0.19 mmol, 0.035 eq) and K₂CO₃ (2 M in water, 3.34 mL, 6.67 mmol, 1.23 eq) at room temperature. The reaction mixture was heated to 80° C. for 16 h before it was diluted with water and the product was extracted with ethyl acetate (3×15 mL).

The combined organic extracts were dried over Na2SO4, the solvent was removed under reduced pressure and the product was purified by flash chromatography (4-34% EtOAc linear gradient in hexanes) to give S5 (1.06 g, 83%) as a white solid.

¹HNMR (400 MHz, CDCl₃) δ=7.48-7.38; (m, 2H), 7.25-7.18; (m, 2H), 6.69; (d, J=3.6 Hz, 1H), 3.91; (s, 3H), 2.29; (d, J=2.0 Hz, 3H).

¹³C NMR (101 MHz, CDCl₃) δ=161.58; (d, J=244.9 Hz), 159.23, 156.61; (d, J=3.0 Hz), 143.71, 132.01; (d, J=5.5 Hz), 129.07; (d, J=8.5 Hz), 125.96; (d, J=17.5 Hz), 120.37; (d, J=3.3 Hz), 120.12, 111.52; (d, J=24.7 Hz), 107.15, 52.02, 14.66; (d, J=3.5 Hz).

MS (ESI) for C₁₃H₁₁FO₃ [M+H]⁺ 235.09.

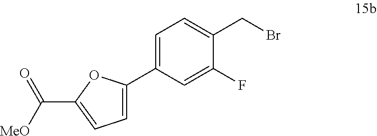 [SMILES: COC(=O)c1ccc(-c2ccc(CBr)c(F)c2)o1]

Azobisisobutyronitrile (35 mg, 0.213 mmol, 0.1 eq) and N-bromosuccinimide (416 mg, 2.34 mmol, 1.1 eq) were added to a solution of S5 (500 mg, 2.13 mmol, 1.0 eq) in 23 mL CCl₄ and the reaction mixture was stirred for 12 h at 96° C. The yellow suspension was filtered and the solvent was removed under reduced pressure. The crude product was purified by flash chromatography (4-34% EtOAc linear gradient in hexanes) providing 15b (425 mg, 64%) as a slightly yellow solid.

¹H NMR (400 MHz, CDCl₃) δ=7.52; (dd, J=8.0, 1.7 Hz, 1H), 7.46; (dd, J=10.6, 1.7 Hz, 1H), 7.42; (t, J=8.0 Hz, 1H), 7.23; (d, J=3.6 Hz, 1H), 6.76; (d, J=3.6 Hz, 1H), 4.51; (d, J=1.1 Hz, 2H), 3.91; (s, 3H).

¹³C NMR (101 MHz, CDCl₃) δ=160.94; (d, J=250.4 Hz), 159.07, 155.60; (d, J=2.9 Hz), 144.31, 131.89; (d, J=8.1 Hz) 131.87; (d, J=3.7 Hz), 125.73; (d, J=14.9 Hz), 120.87; (d, J=3.5 Hz), 120.01, 112.13; (d, J=24.0 Hz), 108.37, 52.12, 25.38; (d, J=4.3 Hz).

MS (ESI) for C₁₃H₁₀BrFO₃ [M+H]⁺ 312.93.

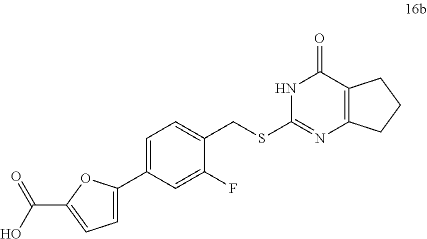 [SMILES: O=C(O)c1ccc(-c2ccc(CSc3nc4c(c(=O)[nH]3)CCC4)c(F)c2)o1]

A suspension of 8 (1.33 g, 7.91 mmol, 1.2 eq) and triethylamine (1.32 mL, 9.49 mmol, 1.2 eq) in 15 mL DMF was stirred for 15 min at room temperature before 15b (2.96 g, 9.49 mmol, 1.0 eq) was added and the reaction mixture was stirred for 16 h at room temperature. The solids were filtered, washed with small amounts of water, methanol and diethyl ether, and the product was dried under vacuum to give the corresponding methyl ester (2.98 g, 94%) as a white solid. ¹H NMR (400 MHz, DMSO) δ=12.56; (bs, 1H), 7.69-7.56; (m, 3H), 7.42; (d, J=3.7, 1H), 7.25; (d, J=3.7H), 4.42; (s, 2H), 3.83; (s, 3H), 2.81-2.71; (m, 2H), 2.62-2.54; (m, 2H), 2.01-1.89; (m, 2H), MS (ESI) for C₂₀H₁₇FN₂O₄S [M+H]⁺ 401.05.

An aqueous 1 M solution of sodium hydroxide (4.12 mL, 4.12 mmol, 3.2 eq) was added to the methyl ester (515 mg, 1.29 mmol, 1.0 eq) in 13 mL of a 2:1 mixture of THF and methanol and the reaction mixture was heated to 60° C. for 2 h. After cooling down to room temperature the mixture was diluted with 3 mL water and acidified with 1 M HCl (pH˜2, ca. 4.5 mL). The precipitated product was filtered and washed with water providing 16b (490 mg, 98%) as a white solid.

¹H NMR (400 MHz, DMSO) δ=12.83; (s, 2H), 7.67-7.55; (m, 3H), 7.32; (d, J=3.6 Hz, 1H), 7.21; (d, J=3.6 Hz, 1H), 4.42; (s, 2H), 2.83-2.71; (m, 2H), 2.61-2.53; (m, 2H), 2.03-1.87; (m, 2H).

¹³C NMR (101 MHz, DMSO) δ=168.70, 160.84, 160.72; (d, J=246.4 Hz), 159.16, 154.63; (d, J=2.9 Hz), 144.55, 132.31; (d, J=4.3 Hz), 130.45; (d, J=8.9 Hz), 124.86; (d, J=14.8 Hz), 120.20; (d, J=3.3 Hz), 119.83, 119.41, 111.15; (d, J=24.0 Hz), 109.18, 34.18, 27.13, 26.70, 20.56.

MS (EST) for C₁₉H₁₅FN₂O₄S [M+H]⁺ 387.15.

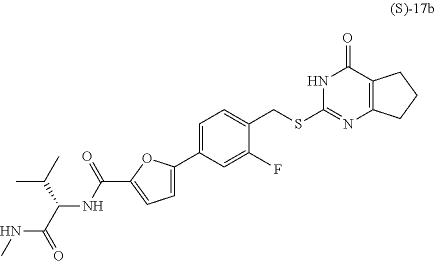 [SMILES: CNC(=O)[C@@H](NC(=O)c1ccc(-c2ccc(CSc3nc4c(c(=O)[nH]3)CCC4)c(F)c2)o1)C(C)C]

To a solution of 16b (60 mg, 0.155 mmol, 1.0 eq), EDCI⋅HCl (45 mg, 0.233 mmol, 1.5 eq), HOBt (31 mg, 0.233 mmol, 1.5 eq) and DIPEA (40 ∛L, 0.233 mmol, 1.5 eq) in 1 mL DMF was added S4 (76 mg, 0.31 mmol, 2,0 eq). The reaction mixture was stirred for 4 h at room temperature before it was diluted with ethyl acetate (ca. 5 mL) and washed with 0.1M HCl (2×10 mL). The aqueous phase was extracted with ethyl acetate (3×10 mL) and the combined organic extracts were washed with an aqueous saturated solution of NaHCO₃ (1×10 mL) and brine (1×10 mL).

The organic phase was dried over Na₂SO₄ and the solvent was removed under reduced pressure. The crude product was purified by flash chromatography (0-10% methanol linear gradient in DCM) and preparative HPLC (linear gradient 10-100% acetonitrile/MeOH=1:1, 0.1% TFA, 10 min) providing (S)-17b (61 mg, 79%) as a white solid.

¹H NMR (400 MHz, DMSO) δ=12.59; (bs, 1H), 8.40; (d, Hz, 1H), 8.06; (q, J=4.5 Hz, 1H), 7.86; (dd, J=11.2, 1.7 Hz, 1H), 7.71; (dd, J=8.0 Hz, 1.7, 1H), 7.60; (t, J=8.0 Hz, 1H), 7.26; (d, J=3.6 Hz, 1H), 7.18; (d, J=3.6 Hz, 1H), 4.43; (s, 2H), 4.20; (t, J=8.8 Hz, 1H), 2.84-2.71; (m, 2H), 2.67-2.54; (m, 5H), 2.21-2.07; (m, 1H), 2.03-1.89; (m, 2H), 0.90; (d, J=6.7 Hz, 3H), 0.88; (d, J=6.7 Hz, 3H).

¹³C NMR (101 MHZ, DMSO) d=171.24, 168,54, 160.80; (d, J=245.9 Hz), 160.62, 157.31, 153.09; (d, J=2.9 Hz), 147.12, 132.05, 130.78; (d, J=9.0 Hz), 124.31; (d, J=14.6 Hz), 120.30, 116.23, 111.18; (d, J=24.0 Hz), 108.92, 58.45, 34.24, 29.90, 27.16, 26.70, 25.42, 20.57, 19.35, 19.04.

IR (thin film) v 3295, 2958, 1737, 1668, 1519, 1315, 1184 cm⁻¹.

HRMS (ESI) calcd. for C₂₅H₂₇FN₄O₄S [M+H]⁺ 499.1737; found 499.1805.

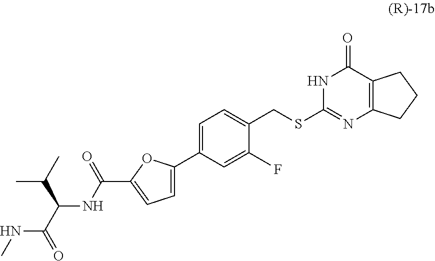 [SMILES: CNC(=O)[C@H](NC(=O)c1ccc(-c2ccc(CSc3nc4c(c(=O)[nH]3)CCC4)c(F)c2)o1)C(C)C]

Compound (R)-17b was synthesized following the same procedure as described for (S)-17b using (R)-S4 as the coupling partner for 16b. Yield: 79%

¹H NMR (400 MHz, DMSO) δ=12.59; (bs, 1H), 8.40; (dJ=8.9 Hz, 1H), 8.06; (q, J=4.5 Hz, 1H), 7.86; (dd, J=11.2, 1.7 Hz, 1H), 7.71; (dd, J=8.0 Hz, 1.7, 1H), 7.60; (t, J=8.0 Hz, 1H), 7.26; (d, J=3.6 Hz, 1H), 7.18; (d, J=3.6 Hz, 1H), 4.43; (s, 2H), 4.20; (t, J=8.8 Hz, 1H), 2.84-2.71; (m, 2H), 2.67-2.54; (m, 5H), 2.21-2.07; (m, 1H), 2.03-1.89; (m, 2H), 0.90; (d, J=6.7 Hz, 3H), 0.88; (d, J=6.7 Hz, 3H).

¹³C NMR (101 MHz, DMSO) δ=171.24, 168.54, 160.80; (d, J=245.9 Hz), 160.62, 157,31, 153.09; (d, J=2.9 Hz), 147.12, 132.05, 130.78; (d, J=9.0 Hz), 124.31; (d, J=11.6 Hz), 120.30, 116.23, 111.18; (d, J=24.0 Hz), 108.92, 58,45, 34,24, 29.90, 27.16, 26.70, 25.42, 20,57, 19.35, 19.04.

HRMS (ESI) calcd. for C₂₅H₂₇FN4O₄S [M+H]⁺ 499.1737; found 499.1809.

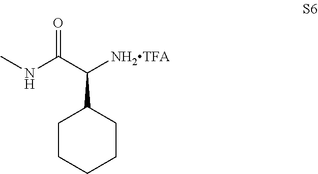

To a suspension of L-cyclohexylglycine (1 g, 6.36 mmol, 1.0 eq) in 10.5 mL water and 5 mL THF were added di-tert-butyl dicarbonate (2.08 g, 9.54 mmol, 1.5 eq) and Na₂CO₃ (1.35 g, 12.72 mmol, 2.0 eq) at room temperature. Further 420 mg (0.3 eq) of di-tert-butyl dicarbonate was added after 12 h as the reaction was not complete (TLC: n-BuOH/conc. AcOH/water=4/1/1, R_{f} (product)=0.35, ninhydrin staining). The reaction mixture was stirred for another 12 h at room temperature before it was quenched by the addition of 2 M HCl (pH˜2). After stirring for another 30 min to hydrolyze unreacted di-tert-butyl dicarbonate the product was extracted with ethyl acetate (3×20 mL). The combined organic extracts were washed with brine, dried over Na₂SO₄ and the solvent was removed under reduced pressure providing Boc-potected L-cyclohexylglycine (1.6 g, 98%) as a yellow oil. The crude product was used for the next step without any further purification. ¹H NMR (400 MHz, CDCl₃) δ=4.99 (d, J=9.0 Hz, 1H), 4.23; (dd, J=9.0, 5.0 Hz, 1H), 1.88-1.57; (m, 6H), 1.45; (s, 9H), 1.23-1.01; (m, 4H).

To a solution of Boc-potected L-cyclohexylglycine (1.6 g, 6.2.2 mmol, 1.0 eq), EDCI⋅HCl (1.43 g, 7.46 mmol, 1.2 eq), and DMAP (100 mg, 0.82. mmol, 0.13 eq) in 63 mL methylene chloride was added methylamine (2M in THF, 3.73 mL, 7.46 mmol, 1.2 eq). The reaction mixture was stirred for 18 h at room temperature before it was transferred in a separatory funnel and washed with 1 M HCl (2×30 mL), aqueous saturated solution of NaHCO₃ (2×30 mL), and brine (1×30 mL).

The organic extracts were dried over Na₂SO₄ and concentrated under reduced pressure to provide tert-butyl-(S)-(1-cyclohexyl-2-(methylamino)-2-oxoethyl)carbamate (1.32 g, 77%) as a yellow oil, which was used for the next step without any further purification. ¹H NMR (400 MHz, CDCl₃) δ=6.02; (bs, 1H), 5.06; (d, J=8.6 Hz, 1H), 3.85; (dd, J=8.6, 6.6 Hz, 1H), 2.81; (d, J=4.9 Hz, 3H), 1.79-1.61; (m, 6H), 1.30-0.89; (m, 4H).

Compound tert-butyl-(5)-(1-cyclohexyl-2-(methylamino)-2-oxoethyl)carbamate (500 mg, 1.85 mmol, 1.0 eq) was dissolved in a 3:1 mixture of methylene chloride and trifluoroacetic acid (20 mL) and stirred for 45 min. The reaction mixture was concentrated under reduced pressure, the remaining yellow oil was re-dissolved in chloroform (ca. 10 mL), and the solvent was again removed in vacuo. This last step was repeated three times to eliminate all traces of trifluoroacetic acid and S6 (TFA salt, 498 mg) was isolated in 99% yield. The NMR of the crude material showed clean product, which was used without any further purification for the next step.

¹H NMR (400 MHz, MeOD) δ=3.55; (d, J=6.4; 1H), 2.862.80; (m, 4H), 1.90-1.66; (m, 6H), 1.36-1.06; (m, 4H).

MS (ESI) for C₉H₁₈N₂O [M+H]⁺ 171.14.

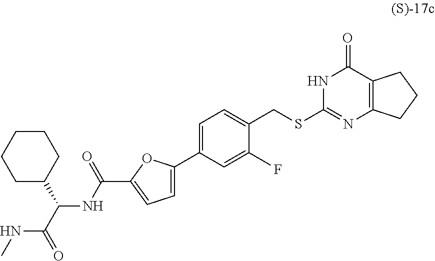 [SMILES: CNC(=O)[C@@H](NC(=O)c1ccc(-c2ccc(CSc3nc4c(c(=O)[nH]3)CCC4)c(F)c2)o1)C1CCCCC1]

16b (70 mg, 0.18 mmol, 1.0 eq) was suspended in 2.2. mL THF and pentafluorophenyl trifluoroacetate (34 μL, 0.198 mmol, 1.1 eq) followed by triethylamine (75 μL, 0.54 mmol, 3.0 eq) were added ad room temperature. After stirring for 2 h S6 (66 mg, 0.234 mmol, 1.3 eq) was added and the reaction mixture was stirred for 18 h at room temperature. Dilution with ethyl acetate (5 mL) and THF (5 mL) was followed by the addition of water (10 mL). The phases were separated and the product was extracted with ethyl acetate (3×15 mL). The combined organic extracts were dried over Na₂SO₄ and the solvent was removed under reduced pressure. The crude product was purified by preparative HPLC (linear gradient 10-100% acetonitrile/MeOH=1:1, 0.1% TFA, 10 min). Lyophilization gave 60 mg (63%) of (S)-17c as a white powder.

¹NMR (400 MHz, DMSO) δ=12.58 (bs, 1H), 8.38; (d, J=8.9 Hz, 1H), 8.08; (q, J=4.5 Hz, 1H), 7.87; (dd, J=11.2, 1.7 Hz, 1H), 7.71; (dd, J=8.0, 1.7 Hz, 1H), 7.60; (t, J=8.0 Hz, 1H), 7.25; (d, J=3.6 Hz, 1H), 7.17; (d, J=3.6 Hz, 1H), 4.43; (s, 2H), 4.25; (t, J=8.9 Hz, 1H), 2.84-2.74; (m, 2H), 2.66-2.55; (m, 5H), 2.03-1.91; (m, 2H), 1.86-1.50; (m, 6H), 1.27-0.88; (m, 5H).

13C NMR (176 MHz, DMSO) δ=171.10, 168.50, 160.84, 160,80 (d, J=245.8 Hz), 157.26, 153.09; (d, J=2.7 Hz), 147.13, 132.03; (d, J=4.2 Hz), 130.79; (d, J=8.9 Hz), 124.30; (d, J=14.8 Hz), 120.31; (d, J=3.2 Hz), 119.58, 116.20, 111.19; (d, J=24.1 Hz), 108.90, 57.50, 38.98, 34.20, 29.36, 28.95, 27.18, 26.70, 25.84, 25.46, 25.39, 20.58.

IR (thin film) v 3289, 2921, 1736, 1668, 1517, 1185, 1167 cm⁻¹.

HRMS (ESI) calcd. for C₂₈H₃₁FN₄O₄S [M+H]⁺ 539.2040; found 539.2109.

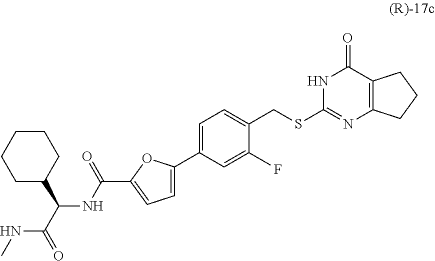 [SMILES: CNC(=O)[C@H](NC(=O)c1ccc(-c2ccc(CSc3nc4c(c(=O)[nH]3)CCC4)c(F)c2)o1)C1CCCCC1]

Compound (R)-17c was synthesized following the same procedure as described for (S)-17c using (R)-S6 as the coupling partner for 16b. Yield: 79%

¹H NMR (400 MHz, DMSO) δ=12.58; (bs, 1H), 8.38; (d, J=8.9 Hz, 1H), 8.08; (q, J=4.5 Hz, 1H), 7.87; (dd, J=11.2, 1.7 Hz, 1H), 7.71; (dd, J=8.0, 1.7 Hz, 1H), 7.60; (t, J=8.0 Hz, 1H), 7.25; (d, J=3.6 Hz, 1H), 7.17; (d, J=3.6 Hz, 1H), 4.43; (s, 2H), 4.25; (t, J=8.9 Hz, 1H), 2.84-2.74; (m, 2H), 2.66-2.55; (m, 5H), 2.03-1.91; (m, 2H), 1.86-1.50; (m, 6H), 1.27-0.88; (m, 5H).

¹³C NMR (176 MHz, DMSO) δ=171.10, 168.50, 160.84, 160.80 (d, J=245.8 Hz), 157.26, 153.09; (d, J=2.7 Hz), 147.13, 132.03; (d, J=4.2 Hz), 130.79; (d, J=8.9 Hz), 124.30; (d, J=14.8 Hz), 120.31; (d, J=3.2 Hz), 119.58, 116.20, 111.19; (d, J=24.1 Hz), 108.90, 57.50, 38.98, 34.20, 29.36, 28.95, 27.18, 26.70, 25.84, 25,46, 25.39, 20.58.

IR (thin film) v 3289, 2921, 1736, 1668, 1517, 1185, 1167 cm^{31 1}.

HRMS (ESI) calcd. for C₂₈H₃₁FN₄O₄S [M+H]^{'} 539.2040; found 539.2124.

MMP-13 enzyme activation: Full-length recombinant human pro-MMP-13 (rhMMP-13) was purchased from R&D Systems (catalog no. 511-MM; Minneapolis, Minn.). MMP-13 was activated by incubating pro-MMP-13 diluted in 100 μL enzyme assay buffer (EAB; 50 mM Tris⋅HCl, pH 7.5, 100 mM NaCl, 10 mM CaCl₂, 0.05% Brij-35) with 1 mM (p-aminophenyl)mercuric acid (APMA) for 2 h at 37° C.² The stock of active MMP-13 was diluted to 384.6 nM and stored at −80° C.

Inhibitor kinetics: Inhibition experiments were conducted as described previously.³ Briefly, fTHP-15, MMP-13, and inhibitor working solutions were prepared in EAB. All reactions were conducted in 384-well black polystyrene plates (Greiner, N.C., catalog no. 784076). To determine the IC₅₀ of each inhibitor, the compounds were screened in 10-point 3-fold dilution dose-response curve format in triplicates.

The assay began by dispensing 5 μL of test compounds in assay buffer followed by 5 μL of MMP-13. The enzyme was allowed to incubate with the test compounds for 30 min at 25° C. The assays were initiated by addition of 5 μL of fTHP-15 or Knight substrate and immediately placed in the microplate reader to record fluorescence.

To determine IC₅₀ values of each compound, the relative fluorescence units (RFU) from wells containing MMP-13, fTHP-15, and inhibitors were plotted vs. no-enzyme and untreated controls. For each compound, RFUs from the linear part of the curve were fitted with a four parameter equation describing a sigmoidal dose-response curve with adjustable baseline using GraphPad Prism® version 11 suite of programs. The IC₅₀ values of the compounds were determined as the concentrations that resulted in 50% enzyme activity when compared to the activity of the control samples (without a compound). These values were generated from fitted curves by solving for the X-intercept at the 50% (inhibition level of Y-intercept using the built-in dose-response model algorithm of GraphPad Prism (LaJolla, Calif.), Hill slopes were also determined.

Determinations of inhibition constants and modalities were conducted by incubating a range of fTHP-15 substrate concentrations (2-25 μM) with 4 nM MMP-13 at room temperature in the presence of varying concentrations of inhibitors. Fluorescence was measured on a BioTek H1 microplate reader using λ_{excitatio}=393 nm and λₑₘᵢₛₛᵢₒₙ=393 nm. Rates of hydrolysis were obtained from plots of fluorescence versus time using data points from only the linear portion of the hydrolysis curve. All kinetic parameters were calculated using GraphPad Prism, version 5.01 (GraphPad Software, Inc., La Jolla, Calif.).

K_{M} values were determined by nonlinear regression analysis using the one-site hyperbolic binding model⁴ and additionally evaluated by linear analysis. All Kᵢ values were determined by nonlinear regression (hyperbolic equation) analysis using the mixed inhibition model, which allows for simultaneous determination of the mechanism of inhibition, The mechanism of inhibition was determined using the “α” parameter derived from a mixed-model inhibition by GraphPad Prism. The mechanism of inhibition was additionally confirmed by Lineweaver-Burke plots.

Selectivity assay: To determine the selectivity of each inhibitor, the compounds were tested against a selected protease panel consisting of MMP-1, MMP-2, MMP-8, MMP-9, and MMP-14. All enzymes were purchased from R&D Systems and activated according to manufacturer's instructions. Upon activation, each enzyme was diluted in EAB to 200 μM and stored at −80° C. until further use. The compounds were screened as described above in 10-point 3-fold dilution dose-response curve format in triplicate utilizing fTHP-15 as substrate except for MMP-1, for which Knight substrate was used².

Type II collagen assay: To assess the potency of probes using a physiologically relevant substrate we tested compounds in an assay utilizing type II collagen (Sigma-Aldrich, St. Louis, Mo., Cat #234184). All experiments were performed in 384-well white microtiter plates. The assay was initiated by dispensing 9 μL of 333 nM type II collagen in EAB. 2 μL of test compounds in EAB were added. Reactions were initiated by addition of 9 μL of 4 nM MMP-13 in EAB. After 22 h of incubation at 37 ° C., the samples were resolved by electrophoresis on a 8% SDS-PAGE gel. The gel was stained with Coomassie Blue and band intensities quantified vs. no-enzyme and untreated controls. For each compound, band intensity data were fitted with a four parameter equation describing a sigmoidal dose-response curve with adjustable baseline using GraphPad Prism® version 11 suite of programs. The IC₅₀ values were generated from fitted curves by solving for X-intercept at the 50% inhibition level of Y-intercept.

### Crystallization, Structure Determination and Refinement

Protein was prepared as previously described.³ Automated screening for crystallization was carried out using the sitting drop vapor-diffusion method with an Art Robbins Instruments Phoenix system in the X-ray Crystallography Core Laboratory at UTHSCSA. MMP-13 inhibitor complexes were prepared in a 1:5 molar ratio of protein to inhibitor prior to mixing 0.2 μL of protein complexes at 10 mg/mL with 0.2 μL of crystallization reagents from commercial screens. MMP13:(S)-10a crystals were obtained from Microlytic (Woburn, Mass.) MCSG-1 screen condition #17 (0.2 M magnesium chloride, 0.1 M Tris⋅HCl pH 8.5, 25% polyethylene glycol 3350) at 22° C. MMP-13:(S)-17a crystals were obtained from Qiagen JCSG Core III screen condition #34 (0.2 M sodium chloride, 0.1 M Tris pH 7.0, 1.0 M sodium citrate) at 4° C. MMP-13:(R)-17a crystals were obtained from Microlytic MCSG-4 screen condition #70 (0.2 M lithium sulfate 0.1 M Tris⋅HCl pH 8.5, 30% polyethylene glycol 4000) at 22° C. MMP-13: 10d crystals were obtained from Qiagen pHClear screen condition #58 (0.1 M HEPES, 1.6 M ammonium sulfate, pH 7.0) at 4° C. All crystals were mounted in undersized nylon loops with excess mother liquor wicked off and flash-cooled in liquid nitrogen prior to data collection. The structures were determined by the molecular replacement method implemented in PHASER⁵ using PDB entry 4L19 as the search model. Coordinates for all models were refined using PHENIX⁶, including simulated annealing with torsion angle dynamics, and alternated with manual rebuilding using COOT⁷. Non-crystallographic symmetry restraints were used in the refinement of the MMP-13: 10d complex. Data were collected at the Advanced Photon Source NE-CAT beamline 24-ID-E and integrated and scaled using XDS⁸. Data collection and refinement statistics are shown in Table 1. Figures were generated using PyMOL (http://www.pymol.org)⁹.

### DOCUMENTS CITED

(1) Freire, F.; Fisk, J. D.; Peoples, A. J.; Ivancic, M.; Guzei, I. A.; Gellman, S. H. J. Am. Chem. Soc. 2008, 130, 7839.
(2) Knauper, V.; López-Otin, C.; Smith, B.; Knight, G.; Murphy, G. J. Biol. Chem. 1996, 271, 1544.
(3) Spicer, T. P.; Jiang, J.; Taylor, A. B.; Choi, J. Y.; Hart, P. J,; Roush, W. R.; Fields, G. B.; Hodder, P. S.; Minond, D. J. Med. Chem. 2014, 57, 9598.
(4) Copeland, R. A. In Evaluation of Enzyme Inhibitors in Drug Discovery; Copeland, R. A., Ed.; John Wiley & Sons, Inc.: Hoboken, N.J., 2005, p 82.
(5) McCoy, A. J.; Grosse-Kunstleve, R. W.; Adams, P. D.; Winn, M. D.; Storoni, L. C.; Read, R. J. Journal of applied crystallography 2007, 40, 658.

(6) Adams, P. D.; Afonine, P. V.; Bunkoczi, G.; Chen, V. B. Davis, I. W.; Echols, N.; Headd, J. J.; Hung, L. W.; Kapral, G. J.; Grosse-Kunstleve, R. W.; McCoy, A. J.; Moriarty, N. W.; Oeffner, R.; Read, R. J.; Richardson, D. C.; Richardson, J. S.; Terwilliger, T. C.; Zwart, P. H. Acta crystallographica. Section D, Biological crystallography 2010, 66, 213.
(7) Emsley, P.; Lohkamp, B.; Scott, W. G.; Cowtan, K. Acta crvstallographica. Section D, Biological crystallography 2010, 66, 486.
(8) Kabsch, W. Acta crystallographica. Section D, Biological crystallography 2010, 66, 125.
(9) Shi, Q. W.; Su, X. :H.; Kiyota, H. Chem. Rev. 2008, 108, 4295.

All patents and publications referred to herein are incorporated by reference herein to the same extent as if each individual publication was specifically and individually indicated to be incorporated by reference in its entirety.

1. A selective matrix metalloproteinase-13 inhibitor of formula 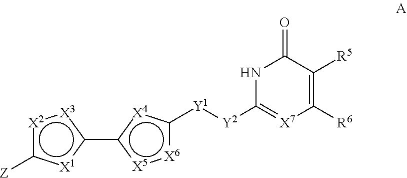
wherein
group Z is of formula S(O)₂NR^{1A}R^{1B} or of formula C(═O )NHCH(R^{2A})C(═O)NHR^{2B};
R^{1A} is H, and R^{1B} is (C₁-C₄)alkyl, HO₂C—(C₁-C₄)alkyl, HO—(C₁-C₄)alkyl, or H₂N—(C₁-C₄)alkyl; or is (C₃-C₇)cycloalkyl, HO₂C—(C₃-C₇)cycloalkyl, HO—(C₃-C₇)cycloalkyl, or H₂N—(C₃-C₇)cycloalkyl; or is 5- to 7-membered heterocyclyl optionally substituted with HO₂C—, HO—, or H₂N—; or is (C₆-C₁₀)aryl optionally substituted with HO₂C—, HO—, or H₂N—; or is 5- to 7-membered heteroaryl optionally substituted with HO₂C—, HO—, or H₂N—;
R^{2A} is (C₁-C₄)alkyl, (C₃-C₇)cycloalkyl, 5- to 7-membered heterocyclyl, (C₆-C₁₀)aryl, or 5- to 7-membered heteroaryl, and R^{2B} is H, (C₁-C₄)alkyl, (C₃-C₇)cycloalkyl, 5- to 7-membered heterocyclyl, (C₆-C₁₀)aryl, or 5- to 7-membered heteroaryl;
X¹ is CH, O, S, C(R³)═C(R³), NR³, or N═C(R³);
X² and X³ are each independently O, S, N or CR³;
such that the ring comprising X¹, X², and X³ is aryl or heteroaryl;
R³ is independently at each occurrence H, (C₁-C₄)alkyl, or halo;
X⁴ is CH, O, S, C(R⁴)═C(R⁴), NR⁴, or N═CR⁴;
X⁵ and X⁶ are each independently O, S, N or CR⁴;
such that the ring comprising X⁴, X⁵, and X⁶ is aryl or heteroaryl;
R⁴ is independently at each occurrence H, (C₁-C₄)alkyl, or halo;
Y^{l} is CHR, O, NR, or a bond;
Y² is S, CHR, NR, or O, or a bond;
X⁷ is N or CR;
R is H or (C₁-C₄)alkyl;
R⁵ and R⁶ are each independently H, (C₁-C₄)alkyl, or halo; or R⁵ and R⁶ together with the ring carbon atoms to which they are bonded together form a 5- to 7-membered cycloalkyl ring or a 5- to 7-member heterocyclyl ring;
or a pharmaceutically acceptable salt thereof.

2. The inhibitor of claim 1 wherein X¹ and X⁴ are CH═CH.

3. The inhibitor of claim 1 wherein X² and X³ are both CR³.

4. The inhibitor of claim 1 wherein X⁵ and X⁶ are both CR⁴.

5. The inhibitor of claim 1 wherein Xⁱ is O.

6. The inhibitor of claim 1 wherein R³ and R⁴ are all H.

7. The inhibitor of claim 1 wherein at least one R⁴ is F.

8. The inhibitor of claim 1 wherein X¹ is CH═CH, and X² and X³ are both CH; and wherein X⁴ is CH═CH, and X⁵ and X⁶ are both CH.

9. The inhibitor of claim 8 wherein the compound is any one of the following: 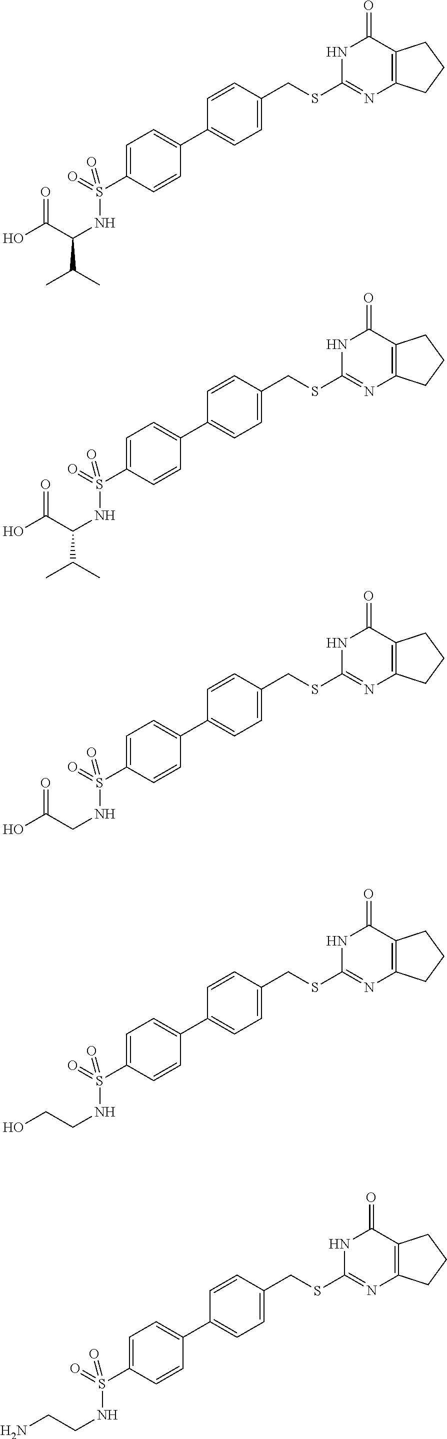 [SMILES: CC(C)[C@H](NS(=O)(=O)c1ccc(-c2ccc(CSc3nc4c(c(=O)[nH]3)CCC4)cc2)cc1)C(=O)O ; CC(C)[C@@H](NS(=O)(=O)c1ccc(-c2ccc(CSc3nc4c(c(=O)[nH]3)CCC4)cc2)cc1)C(=O)O ; O=C(O)CNS(=O)(=O)c1ccc(-c2ccc(CSc3nc4c(c(=O)[nH]3)CCC4)cc2)cc1 ; O=c1[nH]c(SCc2ccc(-c3ccc(S(=O)(=O)NCCO)cc3)cc2)nc2c1CCC2 ; NCCNS(=O)(=O)c1ccc(-c2ccc(CSc3nc4c(c(=O)[nH]3)CCC4)cc2)cc1] 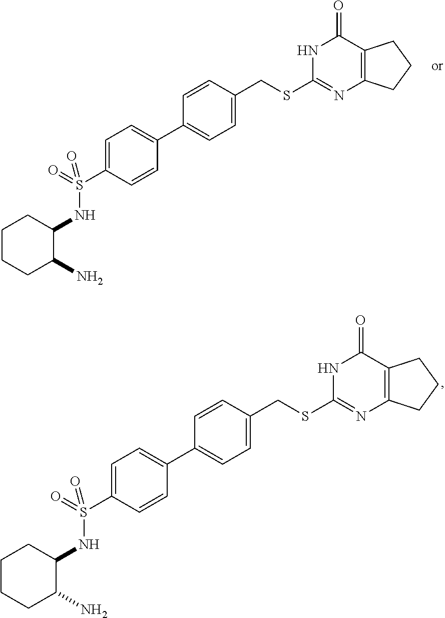
or a pharmaceutically acceptable salt thereof.

10. The inhibitor of claim 1 wherein X¹ is O, X² and X³ are both CH; wherein X⁴ is CH═CH, and X⁵ and X⁶ are both CH; and R⁴ is H or F.

11. The inhibitor of claim 10 wherein the inhibitor is any one of the following: 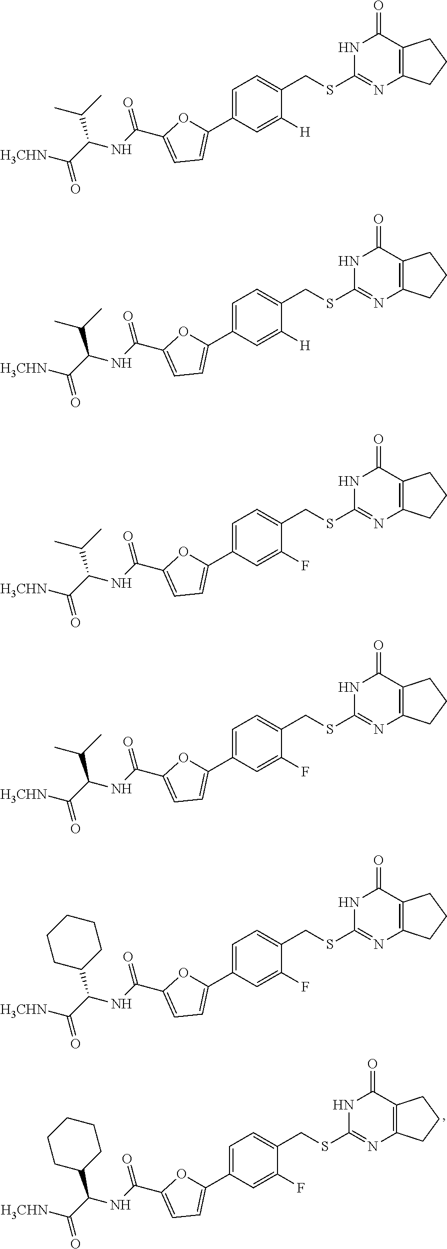
or a pharmaceutically acceptable salt thereof.

12. A pharmaceutical composition comprising an inhibitor of claim 1, and a pharmaceutically acceptable excipient.

13. A method of selective inhibition of matrix metalloproteinase-13 comprising contacting matrix metalloproteinase-13 with an effective amount or concentration of an inhibitor of claim 1.

14. A method of treatment of osteoarthritis, inflammatory bowel disease, melanoma, or breast cancer, comprising administering to a patient afflicted therewith an effective dose of an inhibitor of claim 1.

15-16. (canceled)

17. A pharmaceutical composition comprising an inhibitor of claim 9, and a pharmaceutically acceptable excipient.

18. A method of selective inhibition of matrix metalloproteinase-13 comprising contacting matrix metalloproteinase-13 with an effective amount or concentration of an inhibitor of claim 9.

19. A method of treatment of osteoarthritis, inflammatory bowel disease, melanoma, or breast cancer, comprising administering to a patient afflicted therewith an effective dose of an inhibitor of claim 9.

20. A pharmaceutical composition comprising an inhibitor of claim 11, and a pharmaceutically acceptable excipient.

21. A method of selective inhibition of matrix metalloproteinase-13 comprising contacting matrix metalloproteinase-13 with an effective amount or concentration of an inhibitor of claim 11.

22. A method of treatment of osteoarthritis, inflammatory bowel disease, melanoma, or breast cancer, comprising administering to a patient afflicted therewith an effective dose of an inhibitor of claim 11.
